(19) United States
(12) Patent Application Publication
(10) Pub. No.: US 2026/0298926 A1
(43) Pub. Date: Oct. 1, 2026

(54) METHODS OF TREATING ENDOMETRIOSIS

(71) Applicant: Cedars-Sinai Medical Center, Los Angeles, CA (US)

(72) Inventors: Kate Lawrenson, Los Angeles, CA (US); Marcela Haro, Los Angeles, CA (US)

(73) Assignee: Cedars-Sinai Medical Center, Los Angeles, CA (US)

(21) Appl. No.: 19/477,916

(22) PCT Filed: May 2, 2024
(86) PCT No.: PCT/US2024/027440

§ 371 Date: Oct. 23, 2025

Related U.S. Application Data
(60) Provisional application No. 63/463,516, filed on May 2, 2023.

(51) Int. Cl. G01N 33/569; C12Q 1/6883; G01N 33/68

(52) U.S. Cl. CPC G01N 33/56972; C12Q 1/6883; G01N 33/6854; G01N 33/6869; C12Q 2600/158; G01N 2333/523; G01N 2333/545; G01N 2333/811; G01N 2800/364; G01N 2800/52

(57) ABSTRACT

Methods of treating patients with endometriosis are provided. In various embodiments, the treatment methods select specific types of therapies for patients with different stages of endometriosis detected based on cellular compositions, particularly immune cell compositions, in an endometrial tissue, in combination or alternative to conventional imaging or laparoscopy techniques. Further embodiments provide methods for diagnosing or stratifying patients having peritoneal endometriosis. Overall, heterogeneous immune response underlies subtype-specific and patient-specific heterogeneity in endometriosis.

### CROSS-REFERENCE TO RELATED APPLICATIONS

This application includes a claim of priority under 35 U.S.C. § 119(e) to U.S. provisional patent application No. 63/463,516, filed May 2, 2023, the entirety of which is hereby incorporated by reference.

### FIELD OF INVENTION

This invention relates to assessing and stratifying patients with endometriosis in terms of immune cell phenotypes and immune responses, as well as providing treatments and diagnosis to the patients based on the immune system status.

### BACKGROUND

Endometriosis is characterized by endometrial-type glands and stroma growing outside of the uterine cavity, evading the typical cell-intrinsic and cell-extrinsic safeguards (including immune surveillance) that should prevent cells from surviving at ectopic locations. Chronic inflammation and dysregulation in the immune response contribute to the pathogenesis of endometriosis, however, little is understood regarding how immune dysregulation contributes to the extensive heterogeneity that characterizes endometriosis.

Up to 10% of reproductive-aged women, which is a total of 85 million women in the US at any given time, are affected by endometriosis. Patients can experience a constellation of symptoms including dysmenorrhea, pain associated with intercourse, urination or bowel movements, infertility, nausea, bloating, dysregulated gastrointestinal and bladder function, and fatigue. Symptoms correlate poorly with physical manifestations of disease and there is no way to predict long-term outcomes for patients.

Explaining extensive heterogeneity in symptoms and outcomes of subclasses of endometriosis remains a challenge. The Revised American Society for Reproductive Medicine (r-ASRM) scoring strategy is the most widely used endometriosis stratification scheme in the US and higher-stage endometriosis (III/IV) is associated with greater heritability; but scores do not correlate with fertility, pain, responses to treatment or recurrence. The revised Enzian system addresses a need to describe the features of deep infiltrating endometriosis more fully and may associate with type and severity of symptoms but can only be used for a subset of lesions. The Endometriosis Fertility Index (EFI) combines scores that relate to tubal and ovarian function with age, parity and time trying to conceive to predict pregnancy outcomes although factors that have a strong impact on pregnancy outcome independent of endometriosis likely play a role.

Therefore, it is an object of the present invention to provide methods of assessing and treating patients with endometriosis.

It is another object of the present invention to provide methods of stratifying endometriosis patients and providing respective treatments.

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### SUMMARY OF THE INVENTION

The following embodiments and aspects thereof are described and illustrated in conjunction with compositions and methods which are meant to be exemplary and illustrative, not limiting in scope.

Methods for treating a subject in need thereof are provided, preferably a subject with peritoneal endometriosis, which include: administering one or more of a cluster of differentiation 20 (CD20) inhibitor, an interleukin-17 (IL17) inhibitor, an anti-inflammatory agent, and a therapy to down-regulate central memory T cells, T helper 17 (Th17) T-cells, natural killer cells, B-cells, effector memory T-cells, exhausted T-cells, and/or M2macrophages to a subject indicated as having peritoneal endometriosis with a high density of lymphoid aggregates (LA^{high}). Methods for treating a subject in need thereof are provided, preferably a subject with peritoneal endometriosis, which include: administering an antihistamine and/or a therapy to down-regulate mast cells, MI macrophages, plasma cells, and/or activated T-cells to a subject indicated as having peritoneal endometriosis with a low density of lymphoid aggregates (LA^{low}). Methods for treating a subject in need thereof are also provided, preferably a subject with peritoneal endometriosis, which include: administering one or more of a cluster of differentiation 20 (CD20) inhibitor, an interleukin-17 (IL17) inhibitor, an anti-inflammatory agent, and a therapy to down-regulate central memory T cells, T helper 17 (Th17) T-cells, natural killer cells, B-cells, effector memory T-cells, exhausted T-cells, and/or M2 macrophages to a subject indicated as having peritoneal endometriosis with a high density of lymphoid aggregates (LA^{high}), and/or administering an antihistamine and/or a therapy to down-regulate mast cells, M1 macrophages, plasma cells, and/or activated T-cells to a subject indicated as having peritoneal endometriosis with a low density of lymphoid aggregates (LA^{low}).

In some embodiments, a subject is indicated as having the peritoneal endometriosis with LA^{high} when a number of lymphoid aggregates per unit tissue size in a biological sample of the subject is higher compared to an average lymphoid aggregate number per unit tissue size in a plurality of reference subjects with peritoneal endometriosis. In some embodiments, a subject is indicated as having the peritoneal endometriosis with LA^{high} when a peritoneal endometriosis sample of the subject has 1 or more lymphoid aggregate. In some embodiments, a subject is indicated as having the peritoneal endometriosis with LA^{high} when 3 or more lymphoid aggregates are present in at least 3 different anatomical sites of the subject. In some embodiments, the subject is indicated as having the peritoneal endometriosis with LA^{high} when numbers of central memory T-cells, natural killer (NK) cells, Th17 T cells, effector memory CD8 T cells, Th2 T-cells, exhausted CD8 T-cells and/or macrophages M2 cells per unit tissue size in the biological sample of the subject are higher compared to an average number per unit tissue size of respective cells in the plurality of reference subjects with peritoneal endometriosis.

In some embodiments, a subject is indicated as having the peritoneal endometriosis with LA^{low} when the number of lymphoid aggregates per unit tissue size in the biological sample of the subject is lower compared to the average lymphoid aggregate number per unit tissue size in the plurality of reference subjects with peritoneal endometriosis. In some embodiments, a subject is indicated as having the peritoneal endometriosis with LA^{low} when a peritoneal endometriosis sample of the subject has zero lymphoid aggregate, or no lymphoid aggregate is detectable in the peritoneal endometriosis sample. In some embodiments, a subject is indicated as having the peritoneal endometriosis with LA^{low} when no more than 3 lymphoid aggregates (e.g., 0-3) are present in at least 3 different anatomical sites of the subject. In some embodiments, a subject is indicated as having the peritoneal endometriosis with LA^{low} when numbers of M1 macrophages, mast cells, and/or plasma cells per unit tissue size in the biological sample of the subject are higher compared to the average number per unit tissue size of respective cells in the plurality of reference subjects with the peritoneal endometriosis.

Methods are also provided for treating a subject in need thereof, preferably a subject with endometrioma, which include: administering one of a CD20 inhibitor, an IL17 inhibitor, an anti-inflammatory agent, and optionally further administering a programmed cell death protein 1 (PD1) inhibitor, a cytotoxic T-lymphocyte associated protein 4 (CTLA4) inhibitor, a T cell immunoreceptor with immunoglobulin and ITIM domain (TIGIT) inhibitor, a lymphocyte activation gene 3 (LAG3) inhibitor, a CD86 inhibitor, a nectin-2 inhibitor, a macrophage migration inhibitory factor (MIF) inhibitor, and a tumor necrosis factor (TNF) inhibitor, to a subject indicated as having endometrioma. Methods are also provided for treating a subject in need thereof, which include: administering two or more of a CD20 inhibitor, an IL17 inhibitor, an anti-inflammatory agent, and optionally further administering a programmed cell death protein 1 (PD1) inhibitor, a cytotoxic T-lymphocyte associated protein 4 (CTLA4) inhibitor, a T cell immunoreceptor with immunoglobulin and ITIM domain (TIGIT) inhibitor, a lymphocyte activation gene 3 (LAG3) inhibitor, a CD86 inhibitor, a nectin-2 inhibitor, a macrophage migration inhibitory factor (MIF) inhibitor, and a tumor necrosis factor (TNF) inhibitor, to a subject indicated as having endometrioma.

In some embodiments, a subject is indicated as having endometrioma when a biological sample comprising endometrial-type tissue of the subject is detected with: a higher number of exhausted CD8 T-cells and/or a higher number of plasma cells, per unit tissue size, compared to that in a specimen of peritoneal endometriosis, eutopic endometrium, or uninvolved ovary. In some embodiments, a subject is indicated as having endometrioma when a biological sample comprising endometrial-type tissue of the subject is detected with: a lower number of lymphoid aggregates and/or a lower number of B cells, per unit tissue size, compared to the specimen of peritoneal endometriosis, eutopic endometrium, or uninvolved ovary. In some embodiments, a subject is indicated as having endometrioma when a biological sample comprising endometrial-type tissue of the subject is detected with: a higher number of exhausted CD8 T-cells and/or a higher number of plasma cells, per unit tissue size, compared to that in a specimen of peritoneal endometriosis, eutopic endometrium, or uninvolved ovary; and a lower number of lymphoid aggregates and/or a lower number of B cells, per unit tissue size, compared to the specimen of peritoneal endometriosis, eutopic endometrium, or uninvolved ovary.

In some embodiments, a subject is indicated as having endometrioma when the subject is detected with presence of endometrioma in ovary. Endometrioma can be detected in ovary via laparoscopy, ultrasound, or magnetic resonance imaging (MRI).

In some embodiments, the exhausted CD8 T-cells in a subject with endometrioma are positive for all or one or more of PD-1, CTLA4, TIGIT, V-domain Ig suppressor of T cell activation (VISTA), and LAG3, and are negative for T cell immunoglobulin and mucin domain-containing protein 3 (TIM3). In some embodiments, the macrophages in a subject with endometrioma are M2 macrophages or are positive for all or one or more of IL10, V-set immunoglobulin-domain-containing 4 (VSIG4), regulator of G protein signaling 1 (RGS1), Lymphatic Vessel Endothelial Hyaluronan Receptor-1 (LYVE1), thrombospondin-1 (THBS1), and heparin-binding EGF-like growth factor (HBEGF). In some embodiments, the B-cells in a subject with endometrioma have a lower expression level of membrane spanning 4-domains A1 (MS4A1) and/or CD79A compared to B-cells in the specimen of peritoneal endometriosis, eutopic endometrium, or uninvolved ovary.

In some embodiments, expression levels are measured by single cell RNA sequencing. In some embodiments, expression levels and/or detection of one or more markers are measured with histopathologic assays, optionally including immunohistochemistry, standard histologic stains, and/or non-standard histologic stains.

Methods are also provided for treating a subject in need thereof, e.g., a subject with endometriosis-peritoneal endometriosis with or without endometrioma, which include: administering an endometriosis therapy to a subject detected with: a higher number of myeloid cells and/or a higher number of B cells, per unit tissue size in a biological sample of the subject, compared to respective number per unit tissue size in a specimen of eutopic endometrium, or unaffected ovary, and/or detected with a higher expression level in macrophages of all or one or two of insulin like growth factor 1 (IGF1), embigin (EMB), and transforming growth factor beta-1 (TGFB1) and/or a higher expression level in B cells of MS4A1, CD79A, or both, compared to the specimen of eutopic endometrium, or unaffected ovary.

In some embodiments, an endometriosis therapy comprises one or more of a CD20 inhibitor, an IL17 inhibitor, an anti-inflammatory agent, a therapy to down-regulate central memory T cells, Th17 T-cells, natural killer cells, B-cells, effector memory T-cells, exhausted T-cells, and/or M2 macrophages, an antihistamine, and/or a therapy to down-regulate, mast cells, MI macrophages, plasma cells, and/or activated T-cells; or the endometriosis therapy comprises a procedure to remove endometriosis tissue.

In some embodiments, a subject with endometriosis is detected in a blood sample with a higher level of IL17, IL32, IL1A, IL1B, SERPINEB3 autoantibody, MTUS2 autoantibody, and/or CCL7 autoantibody than a reference subject without endometriosis.

Methods are further provided for treating a subject with endometriosis (i.e., with peritoneal endometriosis, endometrioma, or both), which include: administering a therapy to the subject detected in a biological sample with a higher number of myeloid cells per unit tissue size, compared to that in an uninvolved ovary tissue or with eutopic endometrium of the same subject or of a different subject, wherein the therapy comprises one or more of a CD20 inhibitor, an IL17 inhibitor, an anti-inflammatory, a therapy to down-regulate central memory T cells, Th17 T-cells, natural killer cells, B-cells, effector memory T-cells, exhausted T-cells, and/or M2 macrophages, an antihistamine, and/or a therapy to down-regulate mast cells, M1 macrophages, plasma cells, and/or activated T-cells; or the therapy comprises a procedure to remove endometriosis tissue.

In some embodiments, a biological sample comprises an endometrial-type tissue and optionally surrounding tissue within 1, 2, 3, 4, or 5 cm from the border of the endometrial-type tissue.

Methods for detecting endometrioma in a subject are provided, which include detecting, in a biological sample comprising endometrial tissue of the subject:
a higher number of exhausted CD8 T-cells and/or a higher number of plasma cells, per unit tissue size, compared to respective number of a reference subject with peritoneal endometriosis in the absence of endometrioma, a reference subject with eutopic endometrium, or an uninvolved ovary tissue of the subject or of either reference subject,
a lower number of lymphoid aggregates and/or a lower number of B cells, per unit tissue size, compared to that of the reference subject with peritoneal endometriosis in the absence of endometrioma, and/or
a lower number of myeloid cells, per unit tissue size, than that of the reference subject with peritoneal endometriosis in the absence of endometrioma, optionally the number of the myeloid cells being higher compared to that of the reference subject with eutopic endometrium or compared to the uninvolved ovary tissue.

In some embodiments, the exhausted CD8 T-cells are positive for all or one or more of PD-1, CTLA4, TIGIT, VISTA, and LAG3, and optionally negative for TIM3; the macrophages in the biological sample are in the M2 state or are positive for all or one or more of IL10, VSIG4, RGS1, LYVE1, THBS1, and HBEGF; and/or the B-cells in the biological sample have a lower expression level of MS4A1, CD79A, or both, compared to B-cells of a subject with peritoneal endometriosis but not endometrioma or a subject with eutopic endometrium.

Methods for detecting peritoneal endometriosis in a subject are provided, which include detecting, in a biological sample comprising endometrial tissue of the subject:
a higher number of myeloid cells and/or a higher number of B cells, per unit tissue size, compared to respective number per unit tissue size in a reference subject with endometrioma, a reference subject with eutopic endometrium, or an uninvolved ovary tissue of the subject or of either reference subject,
a higher number of lymphoid aggregates, per unit tissue size, compared to that of the reference subject with endometrioma, and/or
a higher expression level in macrophages of all or one or two of IGF1, EMB, and TGFB1 and/or a higher expression level in B cells of MS4A1, CD79A, or both, compared to that in the reference subject with endometrioma,
wherein the subject does not have or is not detected with endometrioma.

Methods are further provided for assessing peritoneal endometriosis in a subject, which include quantifying a number of lymphoid aggregates per unit tissue size in a biological sample comprising biopsy of a tissue with suspected endometriosis or of an endometrial tissue of the subject, wherein if the number of the lymphoid aggregates is larger than a reference number, further detecting a higher number of all or one or more of Th17 T-cells, NK cells, B cells, effector memory CD8 T cells, Th2 T-cells, exhausted CD8 T-cells, macrophages M2 cells, and cytotoxic CD8 T-cells, per unit tissue size, in the biological sample compared to respective cell numbers detected in a reference subject whose number of the lymphoid aggregates is smaller than the reference number, or wherein if the number of the lymphoid aggregates is smaller than the reference number, further detecting a higher number of all or one or more of macrophages M1, mast cells, and plasma cells per unit tissue size in the biological sample, compared to respective cell numbers detected in a reference subject whose number of the lymphoid aggregates is larger than the reference number. Preferably the subject in the peritoneal endometriosis assessment method does not have endometrioma.

Methods are also provided for classifying or staging endometriosis in a subject in need thereof, which include: measuring in a biological sample comprising (biopsy of) a tissue with suspected endometriosis or of an endometrial tissue of the subject, one or more of: a number of exhausted CD8 T-cells, a number of plasma cells and/or a number of B-cells, a number of myeloid cells, and a number of lymphoid aggregates. In some embodiments, all of a number of exhausted CD8 T-cells, a number of plasma cells and/or a number of B-cells, and a number of myeloid cells are measured. In some embodiments, a number of lymphoid aggregates is measured. In some embodiments, all of a number of exhausted CD8 T-cells, a number of plasma cells and/or a number of B-cells, and a number of myeloid cells, and a number of lymphoid aggregates are measured.

In some embodiment of the classifying or staging endometriosis methods, a higher number of the exhausted CD8 T-cells, a higher number of the plasma cells, a lower number of the B-cells, a lower number of the myeloid cells, and a lower number of the lymphoid aggregates, per unit tissue size, compared to respective numbers in a reference subject with peritoneal endometriosis but not endometrioma, indicates that the subject has endometrioma or that the endometriosis is at an endometrioma stage.

In some embodiment of the classifying or staging endometriosis methods, a lower number of the exhausted CD8 T-cells, a lower number of the plasma cells, a higher number of the B cells, a higher number of the myeloid cells, and a higher number of the lymphoid aggregates, per unit tissue size, compared to respective numbers in a reference subject with endometrioma, indicates that the subject has peritoneal endometriosis but not endometrioma or that the endometriosis is at a peritoneal stage.

In further embodiments, a subject measured as having the peritoneal endometriosis with a larger number of the lymphoid aggregates per unit tissue size than a reference number is treated with one or more of a CD20 inhibitor, an IL17 inhibitor, an anti-inflammatory agent, and/or a therapy to down-regulate central memory T cells, Th17 T-cells, natural killer cells, B-cells, effector memory T-cells, exhausted T-cells, and/or M2 macrophages.

In further embodiments, the subject measured as having the peritoneal endometriosis with a smaller number of the lymphoid aggregates per unit tissue size than a reference number is treated with an antihistamine and/or a therapy to down-regulate mast cells, M1 macrophages, plasma cells, and/or activated T-cells.

Methods are also provided for assessing, stratifying, or monitoring progression of peritoneal endometriosis in a subject, which include: measuring in a biological sample of the subject a higher number of lymphoid aggregates per unit tissue size compared to an average lymphoid aggregate number per unit tissue size in a plurality of reference subjects with peritoneal endometriosis, and/or measuring in the biological sample a higher number of central memory T-cells, NK cells, Th17 T cells, effector memory CD8 T cells, Th2 T-cells, exhausted CD8 T-cells and/or macrophages M2 cells, per unit tissue size, compared to an average number per unit tissue size of respective cells in the plurality of reference subjects with peritoneal endometriosis; so as to indicate that the subject has a high number of lymphoid aggregates and/or select the subject for a therapy comprising a CD20 inhibitor, an IL17 inhibitor, an anti-inflammatory, or a therapy to down-regulate central memory T cells, Th17 T-cells, natural killer cells, B-cells, effector memory T-cells, exhausted T-cells, and/or M2 macrophages.

Methods for assessing, stratifying, or monitoring progression of peritoneal endometriosis in a subject are provided, which include: measuring in a biological sample of the subject a lower number of lymphoid aggregates per unit tissue size compared to an average lymphoid aggregate number per unit tissue size in a plurality of reference subjects with peritoneal endometriosis, and/or measuring in the biological sample of the subject a higher number of M1 macrophages, mast cells, and/or plasma cells per unit tissue size, compared to an average number per unit tissue size of respective cells in the plurality of reference subjects with the peritoneal endometriosis; so as to indicate that the subject has a low number of lymphoid aggregates and/or select the subject for a therapy comprising antihistamine, or a therapy to down-regulate mast cells, M1 macrophages, plasma cells, and/or activated T-cells.

Methods are further provided for treating a subject with endometriosis, which include: administering a therapy to the subject detected in a biological sample with a higher number of IL32-positive T cells per unit tissue size, compared to that in an uninvolved ovary tissue of the same subject or with eutopic endometrium of a reference subject, wherein the therapy comprises one or more of (i) an IL32/IL32-receptor inhibitor, (ii) a therapy to down-regulate resident memory T cells, central memory T cells, Th17 T-cells, natural killer cells, effector memory T-cells, exhausted T-cells, and/or activated T-cells, and (iii) removal of endometriosis tissue, wherein the biological sample comprises an endometriosis tissue and optionally further comprising surrounding tissue within 1, 2, 3, 4, or 5 cm from the border of the endometriosis tissue. In some embodiments, the higher number of IL-32 positive T cells per unit tissue size is compared to an endometriosis-free biopsy of the subject.

Other features and advantages of the invention will become apparent from the following detailed description, taken in conjunction with the accompanying drawings, which illustrate, by way of example, various features of embodiments of the invention.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 Shows a diagram depicting the workflow utilized in Examples.

FIG. 2 Depicts that immune cell composition differs depending on their environment.

FIG. 3 Depicts the enrichment of exhausted CD8 T-cells in endometrioma.

FIG. 4 depicts the immune checkpoints expression on exhausted CD8 T-cells.

FIG. 5 depicts that macrophages and endometrial epithelial potentially inhibit CD8 T-cells.

FIG. 6 depicts that myeloid cells are enriched in endometriosis.

FIG. 7 depicts that macrophages have different roles in different environments.

FIG. 8 depicts B-cells enriched in endometriosis and plasma cells in endometrioma.

FIG. 9 depicts B-cell recruitment signals in endometriosis and endometrioma samples.

FIG. 10 depicts factors affecting B-cell recruitment (Fonseca M., et al, Nature Genetics 2023).

FIG. 11 depicts that mesothelial cells interact with immune cells in endometriosis.

FIG. 12 depicts comparison of B cells and plasma cells.

FIG. 13 depicts SERPINB3 autoantibodies are elevated in endometriosis patient blood.

FIG. 14 depicts B-cells in endometriosis (GC-like B-cells).

FIG. 15 depicts Lymphoid aggregates (LAs) in endometrioma and peritoneal endometriosis.

FIG. 16 depicts immune cell composition differences between LA^{high} endometriosis and LA^{low}.

FIG. 17 depicts a diagram of experiments to study the impact of blocking B cells (by use of anti-CD20 mAb) on endometriosis pathogenesis.

FIG. 18 is a graph showing endometriosis burden (P/s) over time (days) in mice treated with anti-CD20 (to inhibit B cells), control IgG, or a sham in the study depicted in FIG. 17. Y-axis “P/s” is a measurement of photons normalized by animal size, wherein the cells are luciferase-labeled and so photon emission is a proxy for endometriosis burden.

FIG. 19 depicts a diagram summarizing cellular differences between peritoneal endometriosis and endometrioma.

FIG. 20 depicts IL 32 is highly expressed by epithelial cell subsets in endometriosis.

FIG. 21 depicts that IL32 is highly expressed by stromal cell subsets in endometriosis.

FIG. 22 depicts that IL32 is highly expressed by many T cell subsets in endometriosis.

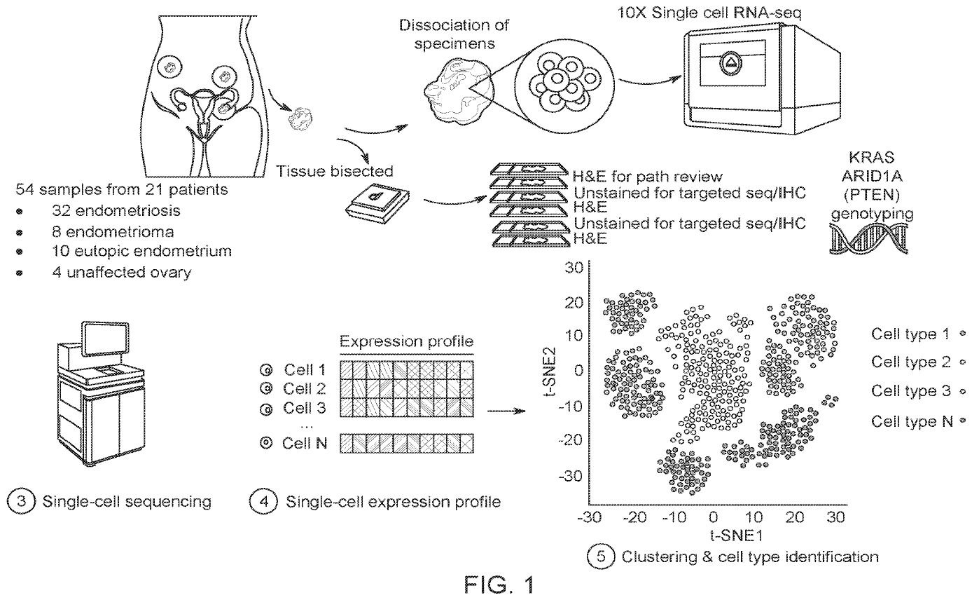

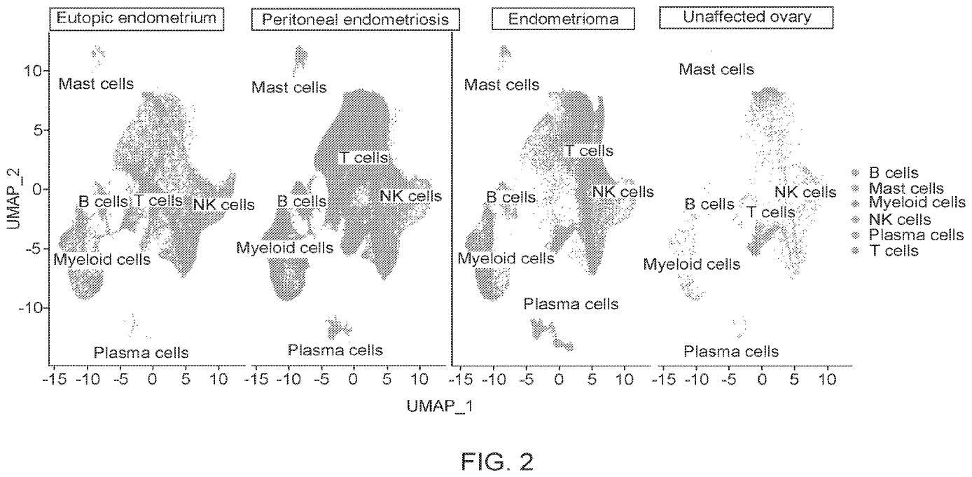

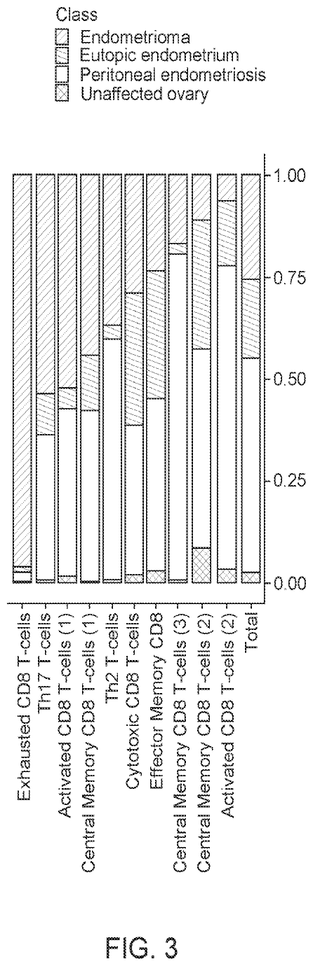

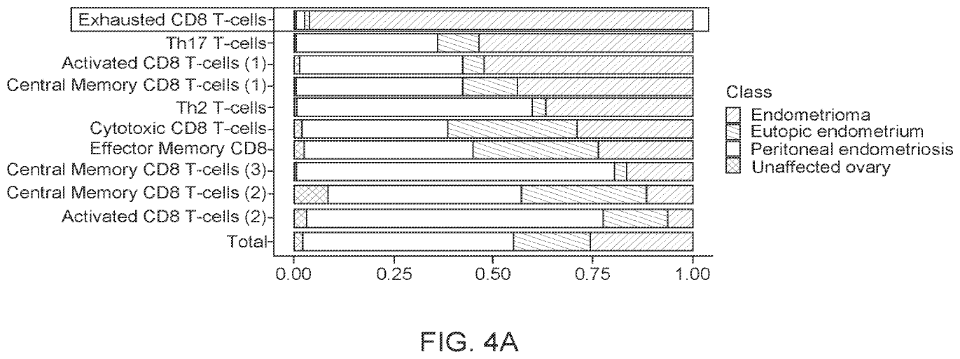

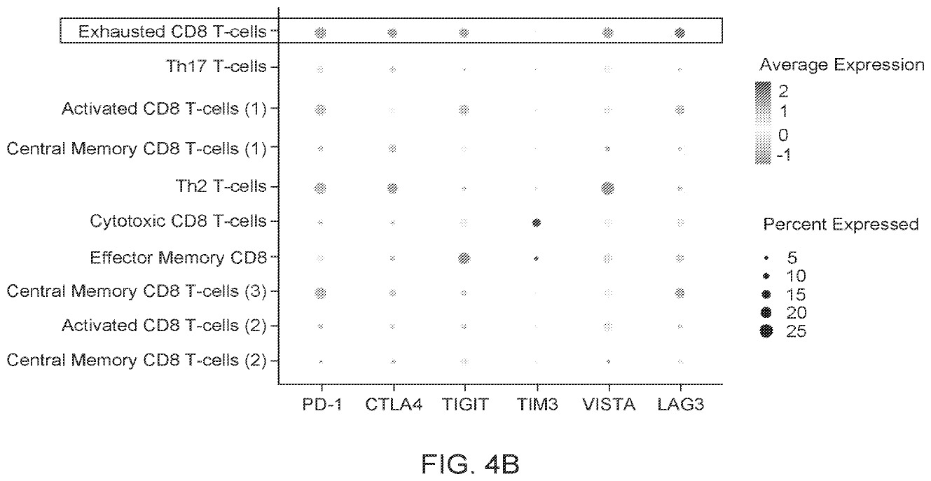

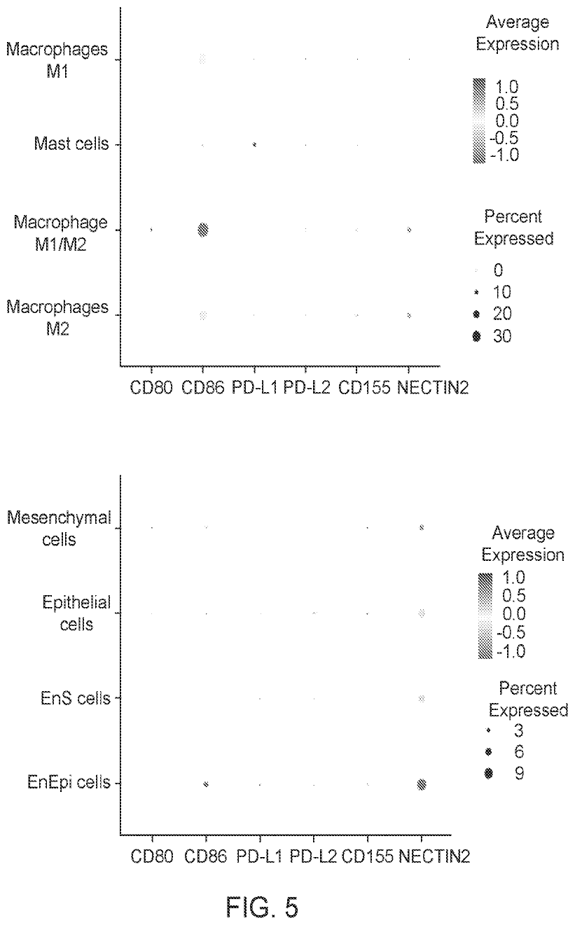

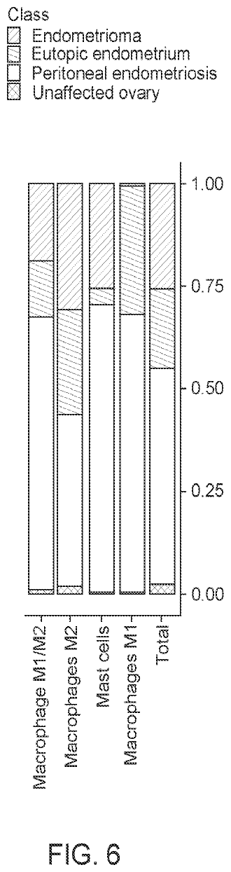

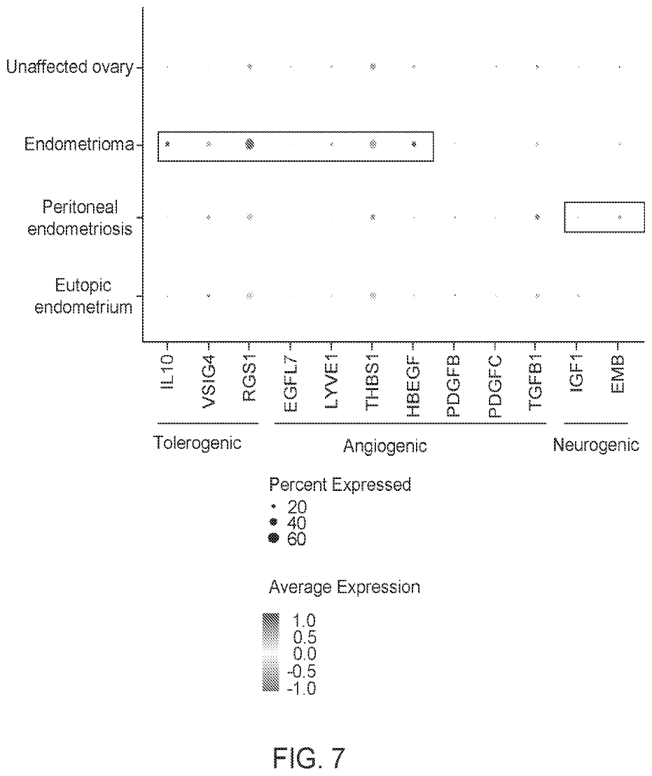

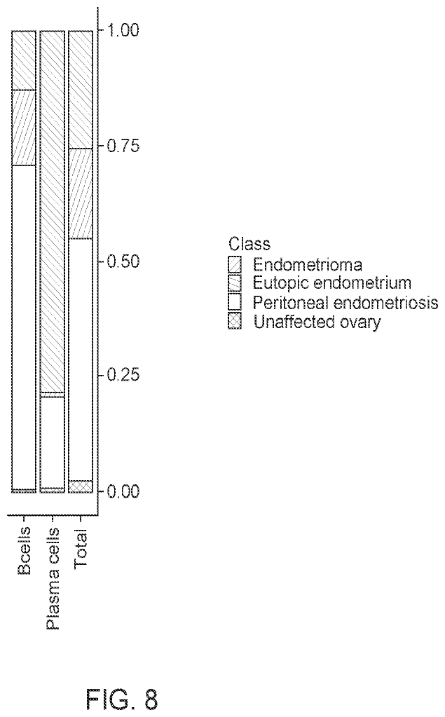

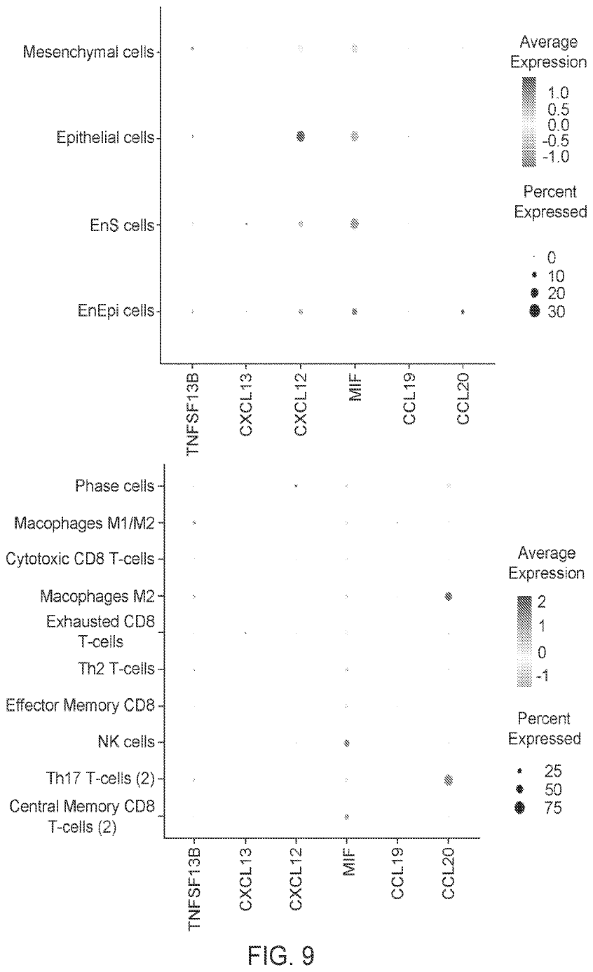

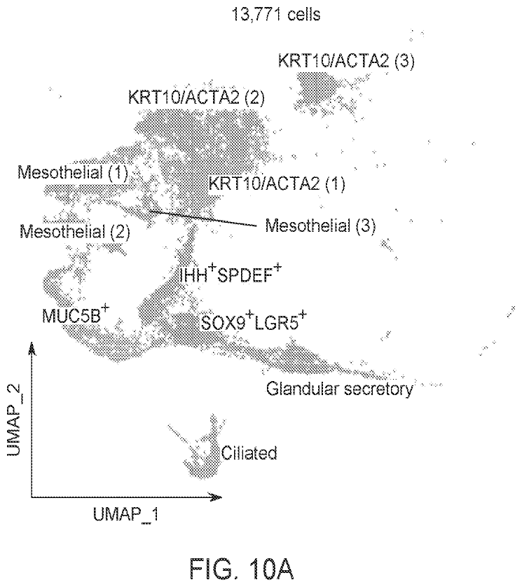

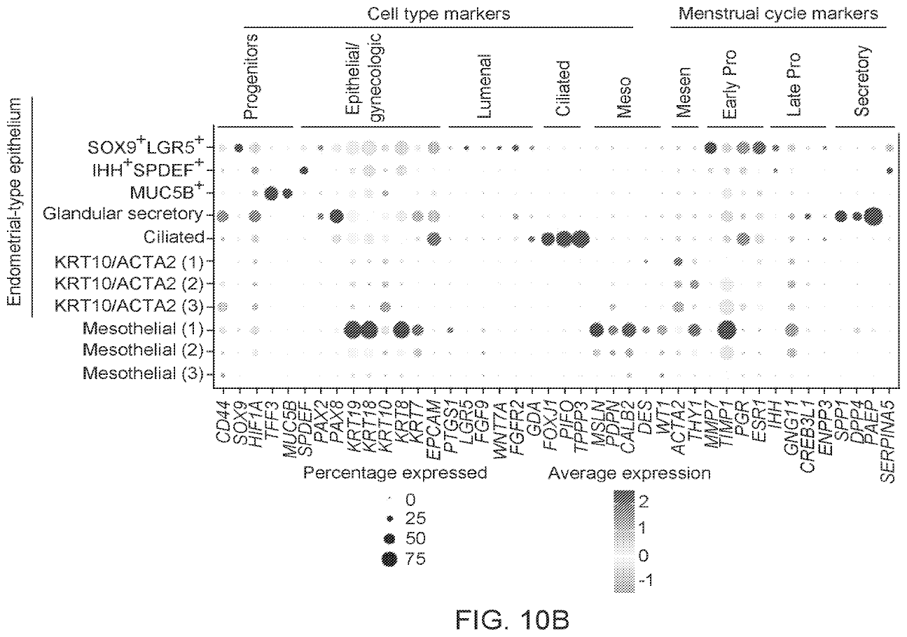

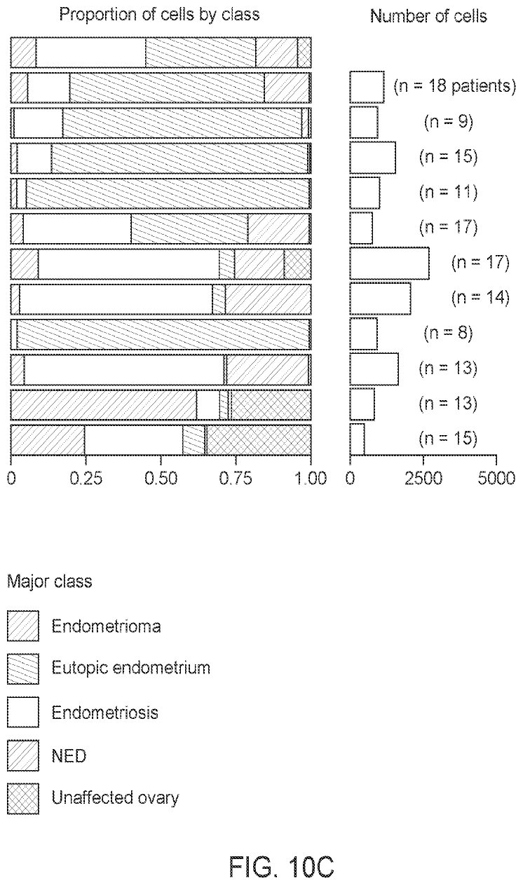

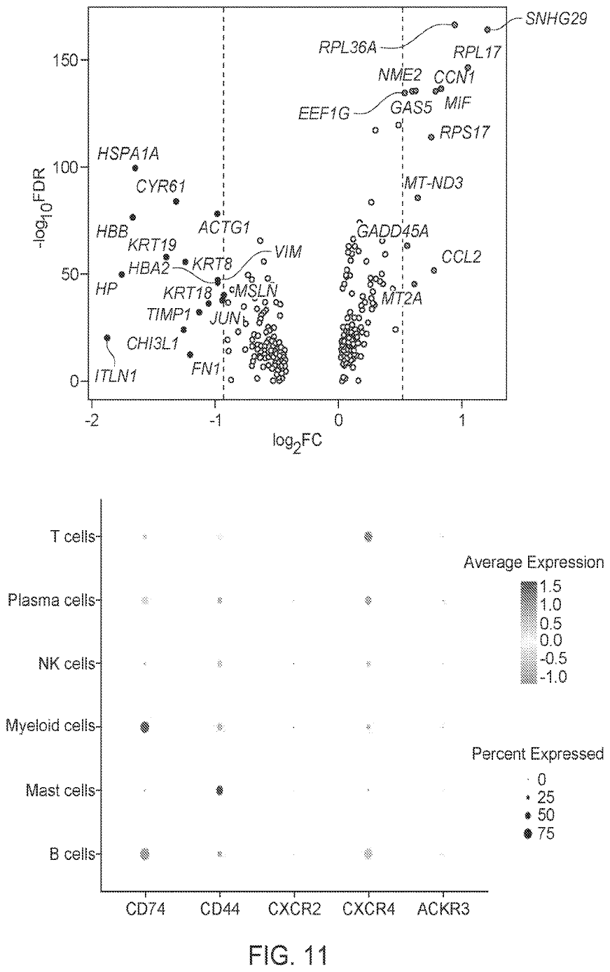

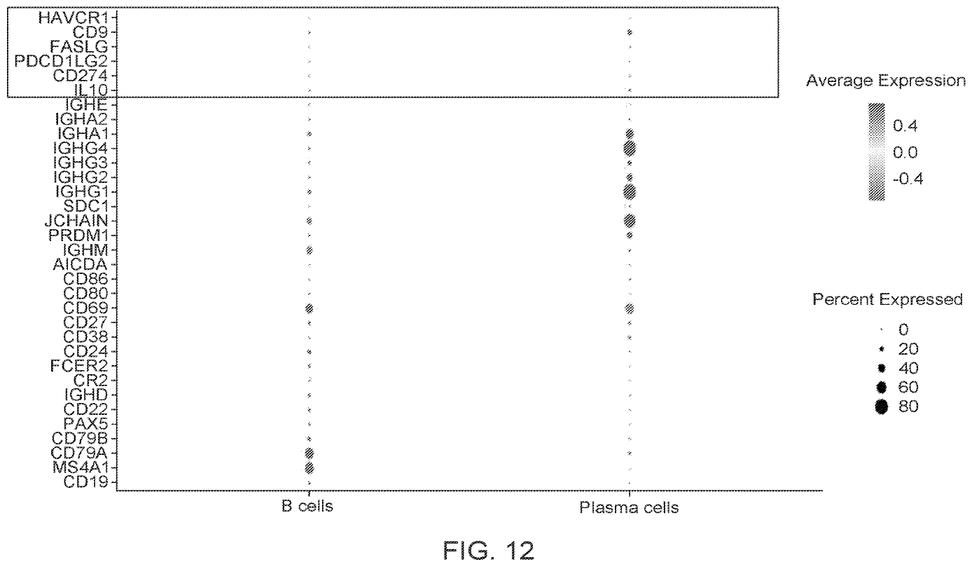

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., Revised, J. Wiley & Sons (New York, NY 2006); March, Advanced Organic Chemistry Reactions, Mechanisms and Structure 7^{th} ed., J. Wiley & Sons (New York, NY 2013); and Sambrook and Russel, Molecular Cloning: A Laboratory Manual 4^{th} ed., Cold Spring Harbor Laboratory Press (Cold Spring Harbor, NY 2012), provide one skilled in the art with a general guide to many of the terms used in the present application. For references on how to prepare antibodies, see D. Lane, Antibodies: A Laboratory Manual 2^{nd} ed. (Cold Spring Harbor Press, Cold Spring Harbor NY, 2013); Kohler and Milstein, (1976) Eur. J. Immunol. 6:511; Queen et al. U.S. Pat. No. 5,585,089; and Riechmann et al., Nature 332:323 (1988); U.S. Pat. No. 4,946,778; Bird, Science 242:423-42 (1988); Huston et al., Proc. Natl. Acad. Sci. USA 85:5879-5883 (1988); Ward et al., Nature 334:544-54 (1989); Tomlinson I. and Holliger P. (2000) Methods Enzymol, 326, 461-479; Holliger P. (2005) Nat. Biotechnol. Sep; 23(9): 1126-36).

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Subject,” “patient,” and “individual” may be used interchangeably. In various embodiments, a subject is a human. In various embodiments, a subject is a female human. In various embodiment, a subject is a biologically female human. In other embodiments, a subject is a mammal, and in some embodiments a female mammal. In some embodiments, a subject is a female human at an age between 14-30, 30-40, or 40-50 years old.

Endometrium is the tissue that lines the uterus.

Endometriosis refers to a condition where endometrial tissue grows outside of the uterus. Endometriosis often occurs on or around reproductive organs in the pelvis or abdomen, including: fallopian tubes, ligaments around the uterus, lining of the pelvic cavity, ovaries, outside surface of the uterus, and space between the uterus and the rectum or bladder; and in rarer instances, it may also grow on or around the bladder, cervix, intestines, rectum, and stomach (abdomen), and vagina or vulva. Endometrial tissue growing in these areas does not shed during a menstrual cycle like healthy endometrial tissue inside the uterus does. The buildup of abnormal tissue outside the uterus can lead to inflammation, scarring and painful cysts. It can also lead to buildup of fibrous tissues between reproductive organs that causes them to “stick” together. Symptoms of endometriosis may include excessive menstrual cramps, abnormal or heavy menstrual flow, and pain during intercourse. Laparoscopy is a minimally invasive surgical procedure that can be used to diagnose endometriosis; and biopsy (e.g., scape off a few cells) is another way to diagnose endometriosis. Imaging such as ultrasound and MRI may be used to look for signs of endometriosis.

Endometrioma is a cystic lesion that develops from endometriosis. Endometrioma is most commonly found in the ovaries. Deep ovarian endometriosis forms dark fluid-filled cavities known as endometriomas. Ovarian endometriomas are filled with menstrual blood.

Therefore, a patient can have endometrioma alone, peritoneal endometriosis alone, or both, i.e., three possible disease states. Further provided herein include stratification methods to stratify peritoneal endometriosis into two mutually exclusive groups, LA^{high} (high number of lymphoid aggregates per unit tissue size, or high density of lymphoid aggregates) or LA^{low} (low number of lymphoid aggregates per unit tissue size, or low density of lymphoid aggregates).

Typically, endometriosis can be broadly categorized into ovarian endometriosis (endometrioma) and peritoneal endometriosis; and the latter may be further categorized into superficial peritoneal endometriosis and deep infiltrating endometriosis (defined clinically as lesions that infiltrate>5 mm under the peritoneal surface). In Examples provided herein, we applied single-cell RNA sequencing (scRNA-seq) to create a cellular atlas of endometriosis and to identify the molecular hallmarks of endometrial-type epithelial and stromal cells in the context of eutopic endometrium (in the correct location within the uterus), endometrioma, or peritoneal endometriosis.

Eutopic endometrium samples may be from subjects with endometriosis, as well as from patients without endometriosis.

Uninvolved ovary tissues may be obtained from subjects with endometriosis, as well as from control, healthy subjects.

“Lymphoid aggregate (LA)” refers to discrete aggregates of lymphoid cells, which can include tertiary lymphoid follicles. These may be found in the uterine endometrium (EM) and also in endometriosis (within or close to the lesion, or at some distance from the lesion). LAs can be present in endometriosis-negative biopsies from a patient with endometriosis located elsewhere in their anatomy. A clinical practitioner can discern or identify LA based on review of histologic slides and/or immunohistological analysis. LAs usually have a B-cell core, which is surrounded by a spheroidal mass of T cells; and the T-cell region is, in turn, surrounded by a diffuse halo of macrophages.

“Biological sample” includes portions of tissues such as biopsy, surgical and autopsy samples, and preserved, and/or frozen sections taken for histologic or other analytical purposes. Such samples include endometrial tissue, the uterine fundus, blood and blood fractions or products (e.g., serum, plasma, platelets, red blood cells, immune cells, stem cells, and the like), sputum, thyroid tissue, cultured cells, e.g., primary cultures, passaged cells, explants, and transformed cells, stool, urine, etc. A biological sample is typically obtained from a eukaryotic organism, most preferably a human or a mammal such as a primate e.g., chimpanzee or human; cow; dog; cat; a rodent, e.g., guinea pig, rat, mouse; or rabbit.

In some embodiments, a biological sample comprise endometrial or endometriosis tissue obtained from a subject. In other embodiments, a biological sample comprises ovarian tissue obtained from a subject.

A “biopsy” refers to the process of removing a tissue sample for diagnostic or prognostic evaluation, and to the tissue specimen itself. Any biopsy technique known in the art can be applied to the diagnostic and prognostic methods of the present invention. The biopsy technique applied will depend on the tissue type to be evaluated (e.g., endometrial, etc.), the size and type of the tissue, among other factors. Representative biopsy techniques include, but are not limited to, excisional biopsy, incisional biopsy, aspirational biopsy, curettage, needle biopsy, surgical biopsy, and bone marrow biopsy. An “excisional biopsy” refers to the removal of an entire endometrial tissue mass with a small margin of non-endometrial tissue surrounding it. An “incisional biopsy” refers to the removal of a wedge of endometrial tissue. Biopsy techniques are discussed, for example, in Harrison's Principles of Internal Medicine, Kasper, et al., eds., 16th ed., 2005, Chapter 70, and throughout Part V.

The terms “overexpress”, “overexpression”, “overexpressed”, “higher expression”, “greater expression”, or “up-regulate(d)” interchangeably refer to a protein or nucleic acid (RNA) that is transcribed or translated at a detectably greater level in comparison to a control. The term includes overexpression due to transcription, post transcriptional processing, translation, post-translational processing, cellular localization (e.g., organelle, cytoplasm, nucleus, cell surface), and RNA and protein stability, as compared to a cell from a woman without endometriosis. Overexpression can be detected using conventional techniques for detecting mRNA (i.e., Q-PCR, RT-PCR, PCR, hybridization, sequencing) or proteins (i.e., ELISA, immunohistochemical and other immunoquantitative or immunolocalization techniques; mass spectrometry, gel electrophoresis). In pair-wise comparisons, overexpression can be 10%, 20%, 30%, 40%, 50%, 60%, 70%, 80%, 90% or more in comparison to a control. In certain instances, overexpression is 1-, 2-, 3-, 4-, 5-, 6-, 7-, 8-, 9-, 10-fold, or more higher levels of transcription or translation in comparison to a control.

The terms “underexpress”, “underexpression”, “underexpressed”, “lower expression”, or “down-regulate(d)” interchangeably refer to a protein or nucleic acid that is transcribed or translated at a detectably lower level in comparison to a control. The term includes down-regulation due to transcription, post transcriptional processing, translation, post-translational processing, cellular localization (e.g., organelle, cytoplasm, nucleus, cell surface), and RNA and protein stability, as compared to a control. Down-regulation can be detected using conventional techniques for detecting mRNA (i.e., Q-PCR, RT-PCR, PCR, hybridization, sequencing) or proteins (i.e., ELISA, immunohistochemical and other immunoquantitative or immunolocalization techniques; mass spectrometry, gel electrophoresis). In pair-wise comparisons, downregulation can be 10%, 20%, 30%, 40%, 50%, 60%, 70%, 80%, 90% or less in comparison to a control or a baseline. In certain instances, downregulation is 1-, 2-, 3-, 4-, 5-, 6-, 7-, 8-, 9-, 10-fold or more lower levels of transcription or translation in comparison to a control or a baseline.

The term “control,” when referring to a traditional pair-wise comparison, refers to a sample from a subject without endometriosis, for example a sample from a healthy subject without endometriosis or other uterine or pelvic conditions, or a sample from a subject having a uterine or pelvic condition or pathology that is not endometriosis. The term control can also refer to a sample from a subject having a different severity of endometriosis. The control can be a reference value that is representative of a population of healthy subjects without endometriosis, or a reference value that is representative of a population of subjects having other uterine conditions or pathologies that are not endometriosis. The control can also be a reference value that is representative of a population of subjects having a different severity of endometriosis. The control can also be from a sample or reference value that is matched to the same menstrual cycle phase as the test sample (e.g., a sample from a subject with endometriosis).

“Therapeutic treatment” refers to chemotherapy, hormonal therapy, other types of pharmacologic therapy, radiotherapy, immunotherapy, and targeted therapies (e.g., biologic, small molecule, pathway or cell cycle inhibitors).

“Inhibitor” in various instances can be a small molecule, peptide, antibody, or a polynucleotide. A “small molecule” or “small organic molecule” refers to an organic molecule, either naturally occurring or synthetic, that has a molecular weight of more than about 50 daltons and less than about 2500 daltons, preferably less than about 2000 daltons, preferably between about 100 to about 1000 daltons, more preferably between about 200 to about 500 daltons. “Antibody” refers to a polypeptide which specifically binds and recognizes an analyte (antigen); and an antibody is often substantially encoded by an immunoglobulin gene or immunoglobulin genes, or antigen binding fragments thereof, or is recombinantly prepared. Antibodies exist, for example as intact immunoglobulins and as a number of well characterized fragments produced by digestion with various peptidases; for instance, Fabs, Fvs, and single-chain Fvs (scFvs). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains. The term also includes genetically engineered forms such as chimeric antibodies (such as humanized murine antibodies), heteroconjugate antibodies such as bispecific antibodies). See also, Pierce Catalog and Handbook, 1994-1995 (Pierce Chemical Co., Rockford, 111.); Kuby, J., Immunology, S. sup. rd Ed., W. H. Freeman & Co., New York, 1997. “Fragment” refers to a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. Inhibitor may also be effectuated by gene silencing, with one or more polynucleotides that knockdown or downregulate transcription, expression or otherwise the level or activity of a target analyte (antigen), and exemplary polynucleotide inhibitors can be RNAi, CRISPR, or siRNA.

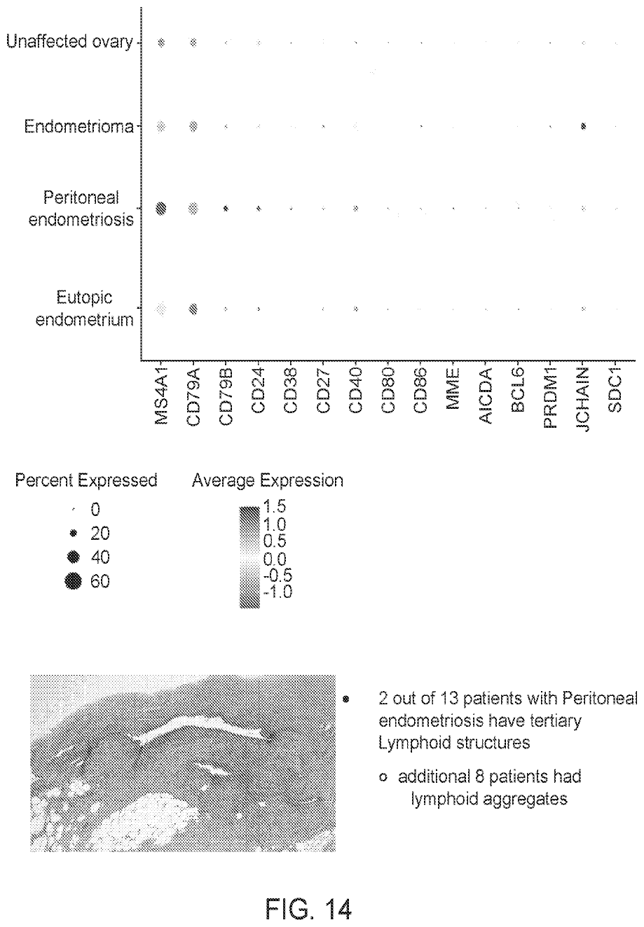

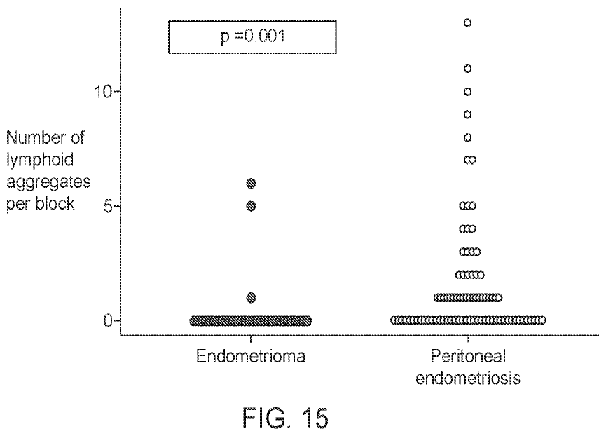

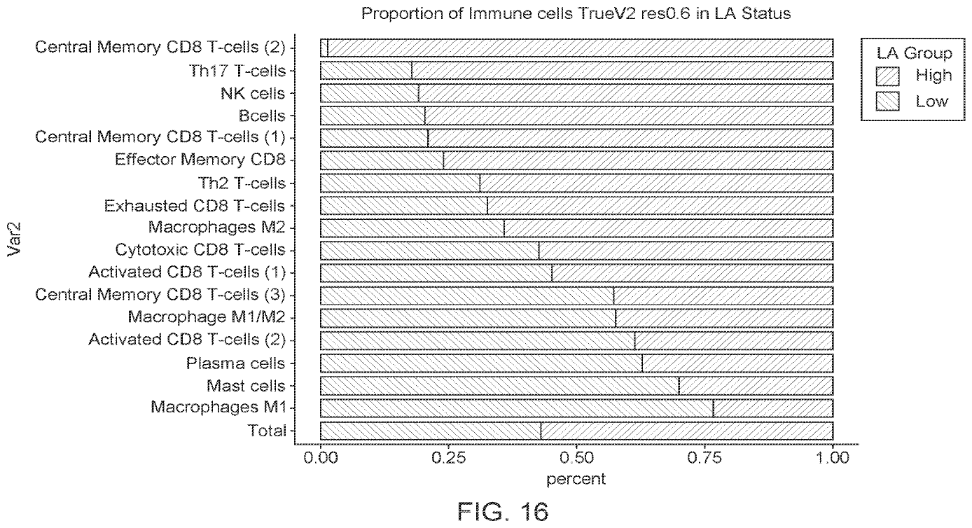

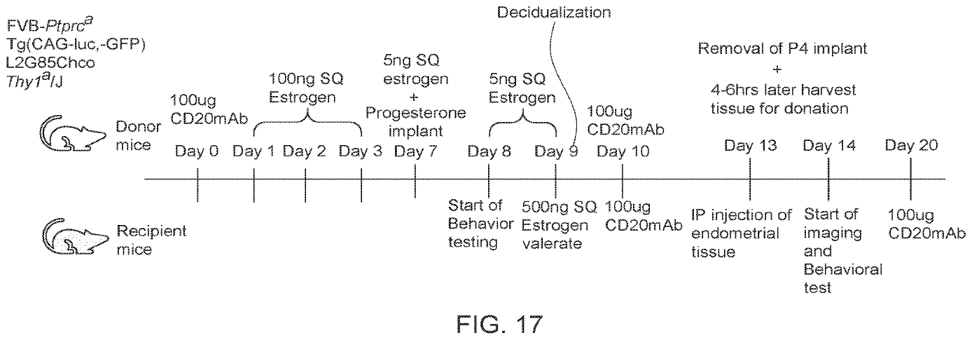

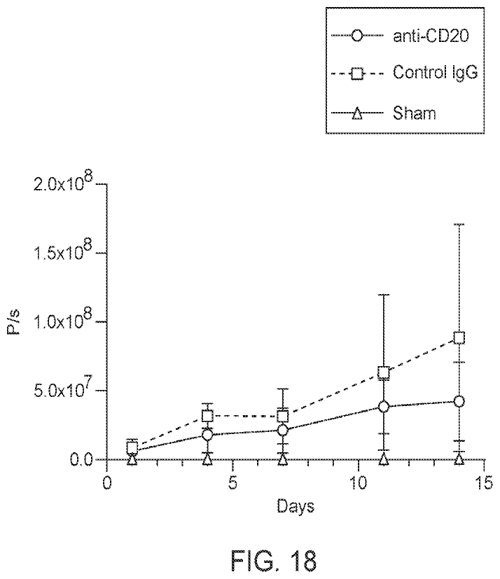

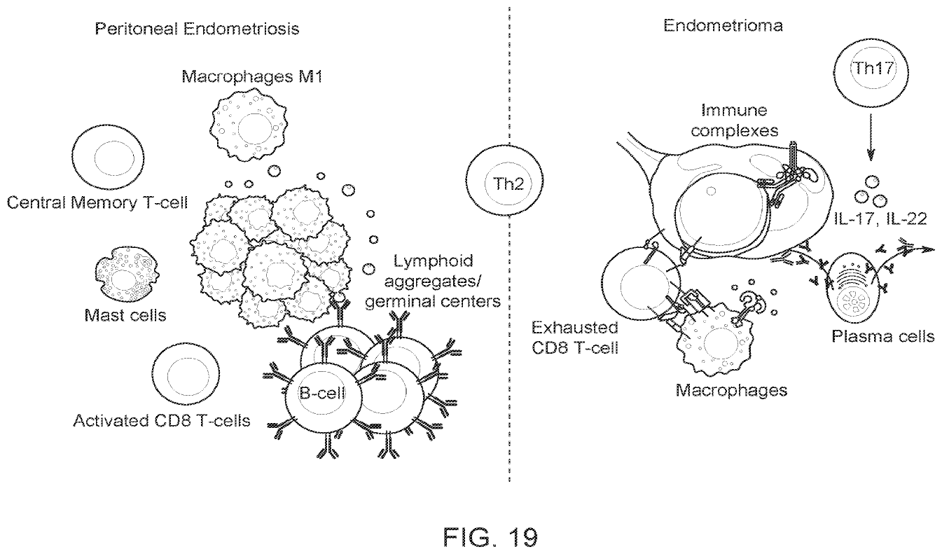

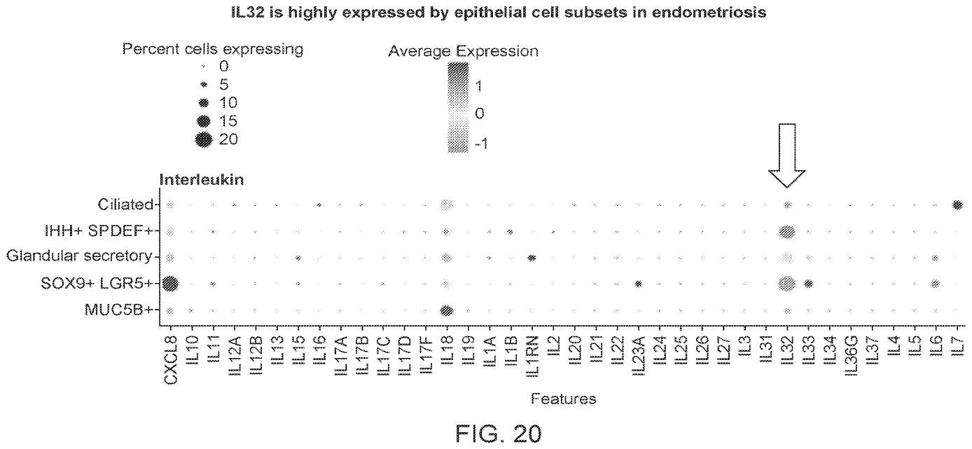

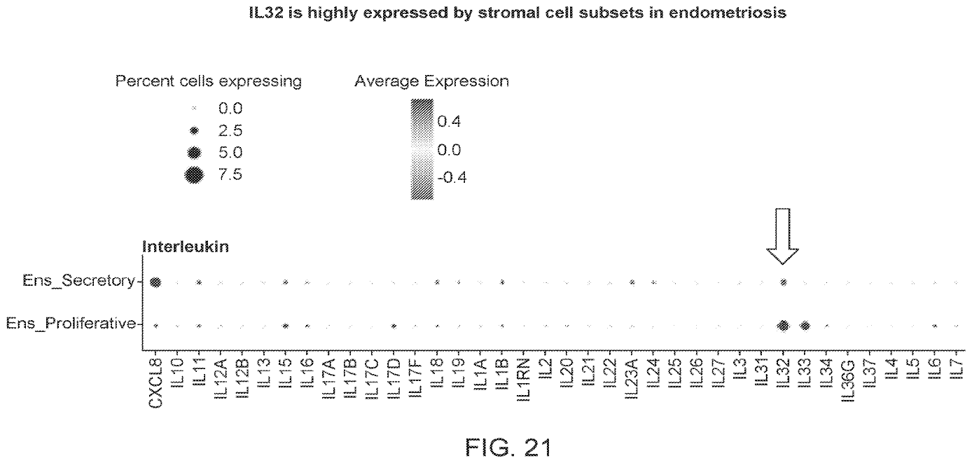

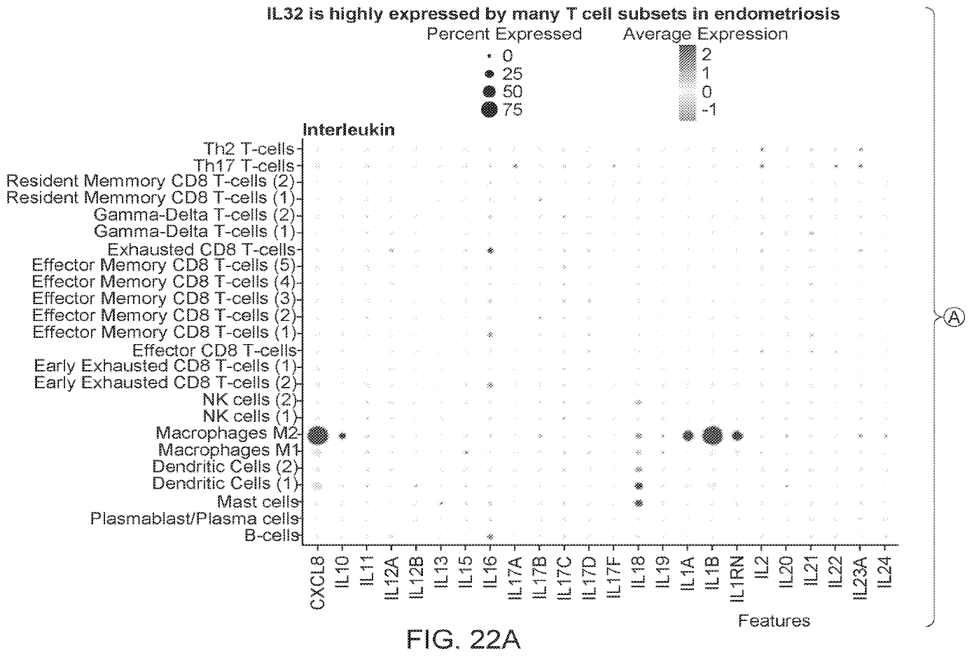

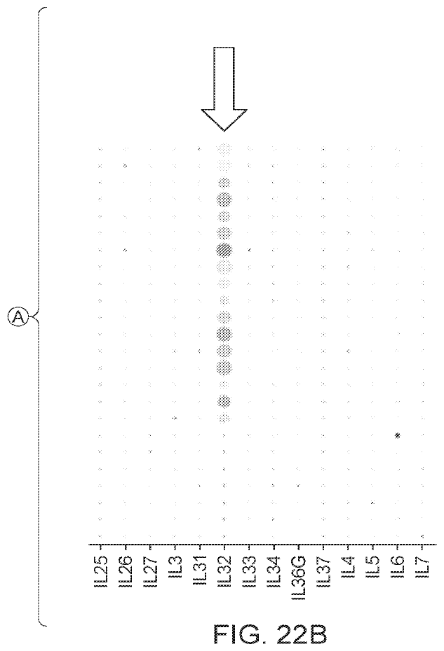

In some embodiments, B cells and plasma cells are each positive (or detectably high) or negative (or detectably low) for different markers. For example, FIG. 14 depicts the positive (or detectably high) and the negative (or detectably low) markers for B cells and for plasma cells.

Herein, we phenotype the immune repertoire in endometriosis and explore the dysregulation in the molecular signals in the tissue microenvironment that influences immune cell recruitment and function. We show that endometriosis patients can be stratified into ‘LA high’ or ‘LA low’ subgroups based on the presence of lymphoid aggregates and germinal centers proximal to lesions. B-cells and plasma cells were particularly enriched in patients with endometriosis, though each differently in peritoneal endometriosis vs. endometrioma. Elevated autoreactive antibodies, B-cell survival and recruitment signals and an interferon signature are seen in endometriosis. Extensive heterogeneity in immune responses to endometriosis may explain, at least in part, the heterogenous presentations and symptoms associated with disease. We also conceive the therapeutic potential of a CD20 inhibitor, e.g., an anti-CD20 monoclonal antibody, for use in patients with endometriosis characterized by elevated B-cell dysfunction.

Endometriosis creates a chronic inflammatory microenvironment that likely contributes to patient symptoms and outcomes. Endometrial-type epithelial cells within lesions secrete chemokines that stimulate immune cell attraction resulting in recruitment of macrophages that infiltrate endometriosis foci. These in turn secrete cytokines that initiate further immune cell recruitment, stimulate stromal cell cytokine secretion and promote the development of a chronic pro-inflammatory state. Therefore, we also conceive targeting immune dysregulation and/or inflammation as a therapeutic strategy for endometriosis, based on understanding of the factors underlying patient heterogeneity in inflammatory phenotypes as disclosed herein.

In some embodiments, endometriosis patients can be stratified into two groups based on its association with lymphoid aggregates (LAs). Number of LAs per unit tissue size, alternatively referred to as density of the LAs, can be measured with quantitative histologic analyses combined with single cell transcriptomics analysis. In some embodiments, the presence (LA^{high} endometriosis) or absence (LA^{low} endometriosis) appears to be a pelvis-wide phenomenon rather than local to endometriosis lesions, indicating that LA status reflects a broader immune reaction, which can be targeted therapeutically. In some embodiments, LA^{high} endometriosis is associated with an enrichment of specific T-cell subsets, and specific inhibitors or antibodies against the T-cell subsets may be used in subjects with LA^{high} endometriosis. In some embodiments, LA^{low} endometriosis is characterized by enrichment of mast cells, and inhibitors against mast cells may be used in subjects with LA^{low} endometriosis.

Various embodiments provide methods of classifying or staging endometriosis in a subject in need thereof, which includes measuring one or more of a number of exhausted CD8 T-cells, a number of plasma cells and/or a number of B-cells, a number of myeloid cells, and a number of lymphoid aggregates, in a biological sample of the subject, said biological sample being or including an endometriosis tissue, an endometriosis-free biopsy or both. In various aspects, endometriosis of LA^{high} and LA^{low} classes are associated with enrichment of different cells, and hence the relative enrichment (or relatively lack thereof) of one or more cell types indicates an endometriosis of different classes, e.g., ovarian endometriosis (endometrioma), or peritoneal endometriosis.

A patient can have endometrioma alone, peritoneal endometriosis alone, or both, i.e., three possible disease states that could be used to guide treatment.

In some embodiments, a patient is one having endometrioma alone. In some embodiments, a patient is diagnosed as having endometrioma alone by one or more methods disclosed herein, and optionally further treated accordingly.

In some embodiments, a patient is one having peritoneal endometriosis alone, i.e., without ovarian endometriosis (endometrioma). In some embodiments, a patient is diagnosed as having peritoneal endometriosis alone by one or more methods disclosed herein, and optionally further treated accordingly.

In some embodiment, a patient is one having both peritoneal endometriosis and endometrioma. In some embodiments, a patient is diagnosed as having both peritoneal endometriosis and endometrioma by one or more methods disclosed herein, and optionally futher treated accordingly.

Diagnosis and/or Treatment for Endometriosis

Various embodiments provide methods for determining or diagnosing a subject with endometriosis.

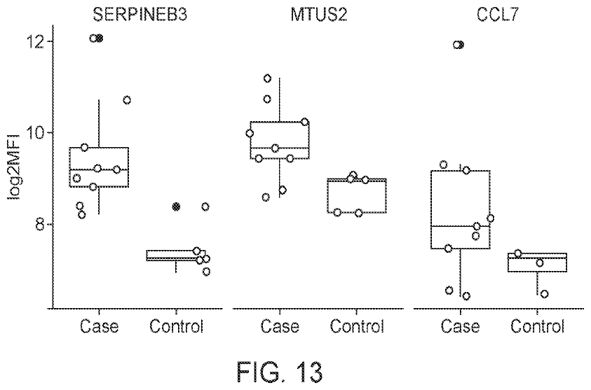

Some embodiments provide that, e.g., shown in FIG. 13, the level of autoantibodies (for SERPINEB3, MTUS2, and/or CCL7) in the blood of endometriosis patients are higher compared to that in blood from women without endometriosis (“control” in FIG. 13). Hence, in some embodiments, a method for diagnosing patients with endometriosis includes measuring a higher level of all or one or more of SERPINEB3 autoantibody (serpinB3 autoantibody), MTUS2 autoantibody (microtubule associated tumor suppressor candidate 2 autoantibody), and CCL7 autoantibody in a biological sample comprising blood or plasma of the subject, compared to that from a biological sample comprising blood or plasma of a reference subject without endometriosis.

In some embodiments, a method for treating a subject in need thereof includes administering one or more endometriosis therapies to a subject detected with a higher level of one or more of SERPINEB3 autoantibody, MTUS2 autoantibody, and CCL7 autoantibody in a biological sample comprising blood or plasma of the subject, compared to that from a biological sample comprising blood or plasma of a subject without endometriosis.

Some embodiments provide a method for diagnosing endometriosis in a subject, which includes measuring a higher number of myeloid cells and/or a higher number of B cells, per unit tissue size in a biological sample of the subject, compared to respective number per unit tissue size in a specimen of eutopic endometrium, or unaffected ovary; preferably the biological sample comprises an endometrial-type tissue.

In some embodiments, a method for treating a subject in need thereof includes administering one or more endometriosis therapies to a subject detected with a higher number of myeloid cells and/or a higher number of B cells, per unit tissue size in a biological sample of the subject, compared to respective number per unit tissue size in a specimen of eutopic endometrium, or unaffected ovary.

Some embodiments provide a method for diagnosing endometriosis in a subject, which includes measuring a higher expression level in macrophages of all or one or two of IGF1, EMB, and TGFB1 and/or a higher expression level in B cells of MS4A1, CD79A, or both in a biological sample of the subject, compared to the specimen of eutopic endometrium, or unaffected ovary; preferably the biological sample comprising an endometrial-type tissue of the subject.

In some embodiments, a method for treating a subject in need thereof includes administering one or more endometriosis therapies to a subject detected with a higher expression level in macrophages of all or one or two of IGF1, EMB, and TGFB1 and/or a higher expression level in B cells of MS4A1, CD79A, or both in a biological sample of the subject, compared to the specimen of eutopic endometrium, or unaffected ovary.

Some embodiments provide a method for diagnosing endometriosis in a subject, which includes measuring a higher number of IL32-positive T cells per unit tissue size in a biological sample of the subject, compared to that in an uninvolved ovary tissue of the same subject or with eutopic endometrium of a reference subject.

In some embodiments, a method for treating a subject in need thereof includes administering an IL32/IL32-receptor inhibitor to a subject detected with a higher number of IL32-positive T cells per unit tissue size in a biological sample of the subject, compared to that in an uninvolved ovary tissue of the same subject or with eutopic endometrium of a reference subject. In some embodiments, a method for treating a subject in need thereof includes administering a therapy to down-regulate resident memory T cells, central memory T cells, Th17 T-cells, natural killer cells, effector memory T-cells, exhausted T-cells, and/or activated T-cells, to a subject detected with a higher number of IL32-positive T cells per unit tissue size in a biological sample of the subject, compared to that in an uninvolved ovary tissue of the same subject or with eutopic endometrium of a reference subject. In some embodiments, a method for treating a subject in need thereof includes removal of endometriosis tissue to a subject detected with a higher number of IL32-positive T cells per unit tissue size in a biological sample of the subject, compared to that in an uninvolved ovary tissue of the same subject or with eutopic endometrium of a reference subject. In some embodiments, a method for treating a subject in need thereof includes performing two or more of (i) administering an IL32/IL32-receptor inhibitor, (ii) administering a therapy to down-regulate resident memory T cells, central memory T cells, Th17 T-cells, natural killer cells, effector memory T-cells, exhausted T-cells, and/or activated T-cells, and (iii) removal of endometriosis tissue, to a subject detected with a higher number of IL32-positive T cells per unit tissue size in a biological sample of the subject, compared to that in an uninvolved ovary tissue of the same subject or with eutopic endometrium of a reference subject.

In various instances, endometriosis therapy comprises a CD20 inhibitor. In some instances, endometriosis therapy comprises an IL17 inhibitor. In some instances, endometriosis therapy comprises an anti-inflammatory agent. In some instances, endometriosis therapy comprises an IL32 inhibitor or IL32-receptor inhibitor. In some instances, endometriosis therapy comprises a therapy to down-regulate central memory T cells, Th17 T-cells, natural killer cells, B-cells, effector memory T-cells, exhausted T-cells, and/or M2 macrophages, an antihistamine. In some instances, endometriosis therapy comprises a therapy to down-regulate, mast cells, M1 macrophages, plasma cells, and/or activated T-cells. In some instances, endometriosis therapy comprises a procedure to remove endometriosis tissue. In other instances, endometriosis therapy comprises a combination of any two or more of a CD20 inhibitor, an IL17 inhibitor, an IL32 or IL32-receptor inhibitor, an anti-inflammatory agent, and laparoscopic cystectomy to remove endometrial tissue.

Diagnosis and/or Treatment for Endometrioma

Various embodiments provide methods for determining or diagnosing a subject with endometrioma. In some embodiments, endometrioma samples have the following features:
a higher number of exhausted CD8 T-cells, Th17 T cells, activated T cells, central memory T-cells and/or a higher number of plasma cells, per unit tissue size, compared to respective number from peritoneal endometriosis patients (e.g., a patient with peritoneal endometriosis alone, or a patient with both peritoneal endometriosis and endometrioma, or an average number from a plurality of patients of (i), or (ii), of (i)+(ii), of (i)+(ii)+(iii), of (i)+(iii), or of (ii)+(iii), wherein (i) is peritoneal endometriosis alone, (ii) is peritoneal endometriosis and endometrioma, and (iii) is endometrioma alone) or compared to uninvolved ovary tissue;
a lower number of lymphoid aggregates and/or a lower number of B cells, per unit tissue size, compared to that of a subject with peritoneal endometriosis; and
myeloid cells with altered marker profiles and function.

In some embodiments, the exhausted CD8 T-cells in patients with endometrioma are positive for, or expressing higher than average, all or one or more of PD-1, CTLA4, TIGIT, VISTA, and LAG3, and optionally negative for, or expressing lower than average, TIM3. In some embodiments, macrophages in patients with endometrioma are positive for, or expressing higher than average, all or one or more of IL10, VSIG4, RGS1, LYVE1, THBS1, and HBEGF. In some embodiments, B-cells in patients with endometrioma have a lower expression level of MS4A1, CD79A, or both, compared to B-cells in peritoneal endometriosis, eutopic endometrium or unaffected ovary.

Some embodiments provide a higher number of the exhausted CD8 T-cells, a higher number of the plasma cells, a lower number of the B-cells, a lower number of the myeloid cells, and/or a lower number of the lymphoid aggregates, compared to respective numbers in a reference subject, indicates/identifies that the subject has endometrioma, which can occur alone or in addition to peritoneal disease. In some embodiments the reference subject in a pair-wise comparison to indicate an endometrioma subject is one indicated/identified to have peritoneal endometriosis. In some embodiments, the reference subject in a pair-wise comparison to indicate an endometrioma subject is a plurality of subjects with peritoneal endometriosis (e.g., as an average number). In some embodiments, the reference subject in a pair-wise comparison to indicate an endometrioma subject is a subject with peritoneal endometriosis but without endometrioma. In other embodiments, the reference subject in a pair-wise comparison to indicate an endometrioma subject is identified/indicated is a subject without endometriosis.

Some embodiments provide a method for diagnosing a subject with endometrioma or determining the subject is likely to have endometrioma, which includes measuring a higher number of exhausted CD8 T-cells (per unit tissue size) in an endometrial-type tissue of the subject, compared to that in a specimen of peritoneal endometriosis, eutopic endometrium, or uninvolved ovary.

In some embodiments, a method for treating a subject in need thereof includes administering an endometrioma therapy to a subject detected with a higher number of exhausted CD8 T-cells (per unit tissue size) in an endometrial-type tissue of the subject, compared to that in a specimen of peritoneal endometriosis, eutopic endometrium, or uninvolved ovary.

Some embodiments provide a method for diagnosing a subject with endometrioma or determining the subject is likely to have endometrioma, which includes measuring a higher number of plasma cells, per unit tissue size, compared to that in a specimen of peritoneal endometriosis, eutopic endometrium, or uninvolved ovary.

In some embodiments, a method for treating a subject in need thereof includes administering an endometrioma therapy to a subject detected with a higher number of plasma cells, per unit tissue size, compared to that in a specimen of peritoneal endometriosis, eutopic endometrium, or uninvolved ovary.

Some embodiments provide a method for diagnosing a subject with endometrioma or determining the subject is likely to have endometrioma, which includes measuring a lower number of lymphoid aggregates per unit tissue size, compared to the specimen of peritoneal endometriosis, eutopic endometrium, or uninvolved ovary.

In some embodiments, a method for treating a subject in need thereof includes administering an endometrioma therapy to a subject detected with a lower number of lymphoid aggregates per unit tissue size, compared to the specimen of peritoneal endometriosis, eutopic endometrium, or uninvolved ovary.

Some embodiments provide a method for diagnosing a subject with endometrioma or determining the subject is likely to have endometrioma, which includes measuring a lower number of B cells, per unit tissue size, compared to the specimen of peritoneal endometriosis, eutopic endometrium, or uninvolved ovary.

In some embodiments, a method for treating a subject in need thereof includes administering an endometrioma therapy to a subject detected with a lower number of B cells, per unit tissue size, compared to the specimen of peritoneal endometriosis, eutopic endometrium, or uninvolved ovary.

In various instances, any of the methods for diagnosing a subject with endometrioma or determining the subject is likely to have endometrioma is accompanied by, or replaced by, laparoscopy, ultrasound, or magnetic resonance imaging (MRI).

In various instances, endometrioma therapies include any one or two or more of a CD20 inhibitor, an IL17 inhibitor, an anti-inflammatory agent, a PD1 inhibitor, a CTLA4 inhibitor, a TIGIT inhibitor, a LAG3 inhibitor, a CD86 inhibitor, a NECTIN2 inhibitor, an MIF inhibitor, and a TNF inhibitor; and/or removal (cystectomy) of endometrioma. In some instances, an endometrioma therapy comprises a CD20 inhibitor. In some instances, an endometrioma therapy comprises an IL17 inhibitor. In some instances, an endometrioma therapy comprises an anti-inflammatory agent. In some instances, an endometrioma therapy comprises a PD1 inhibitor. In some instances, an endometrioma therapy comprises a CTLA4 inhibitor. In some instances, an endometrioma therapy comprises a TIGIT inhibitor. In some instances, an endometrioma therapy comprises a LAG3 inhibitor. In some instances, an endometrioma therapy comprises a CD86 inhibitor. In some instances, an endometrioma therapy comprises a NECTIN2 inhibitor. In some instances, an endometrioma therapy comprises an MIF inhibitor. In some instances, an endometrioma therapy comprises a TNF inhibitor.

Diagnosis and/or Treatment for Peritoneal Endometriosis

In some embodiments, a lower number of the exhausted CD8 T-cells, a lower number of the plasma cells, a higher number of the B cells, a higher number of the myeloid cells, and/or a higher number of the lymphoid aggregates, compared to respective numbers in a reference subject, indicates that the subject has peritoneal endometriosis. Endometrioma may or may not be present in that individual indicated as having the peritoneal endometriosis. In some embodiments, the reference subject in a pair-wise comparison to indicate a peritoneal endometriosis patient is a subject with endometrioma. In some embodiments, the reference subject in a pair-wise comparison to indicate a peritoneal endometriosis patient is a subject without endometriosis.

In various embodiments, the relative enrichment (e.g., higher number) or lack of enrichment (e.g., lower number) is a normalized number per unit tissue size. In some embodiments, the relative enrichment (e.g., higher number) or lack of enrichment (e.g., lower number) is quantified for a same number of cells across different specimens/samples. In some embodiments, the relative enrichment (e.g., higher number) or lack of enrichment (e.g., lower number) is quantified per affected tissue, e.g., an affected tissue is one have any apparent or suspected lesions, optionally with margins, which may be identified and excised with surgery laparoscopically or robotically.

Diagnosis and/or Treatment for LA^{high} or LA^{low} Peritoneal Endometriosis Various embodiments provide that peritoneal endometriosis can be stratified into two mutually exclusive groups, i.e., LA^{high} or LA^{low}, and hence a subject with peritoneal endometriosis may be stratified into either one of the two groups and subject to specific treatment therapies.

In some embodiments, patients with endometriosis (or patients with peritoneal endometriosis with or without endometrioma) are stratified in terms of the number (or normalized number) of the lymphoid aggregates in a biological sample comprising endometrial-type tissue and optionally further the surrounding tissue within 1-5 cm of the border of the endometriosis tissue. In some embodiments, patients with endometriosis (or patients with peritoneal endometriosis with or without endometrioma) are stratified in terms of the number (or normalized number) of the lymphoid aggregates in a unit tissue size/volume, i.e., stratified in terms of a density (or normalized density) of the lymphoid aggregates. In some embodiments, different treatments are provided accordingly.

In some embodiments, a subject sub-classified/stratified/identified as having the peritoneal endometriosis with a larger number of the lymphoid aggregates (e.g., having relative enrichment, quantified per unit tissue size; also coined as LA^{high}) than a reference number or a reference subject is treated with one or more of a CD20 inhibitor, an IL17 inhibitor, and an anti-inflammatory. In some embodiments, a subject sub-classified/stratified/identified as having the peritoneal endometriosis with a larger number of the lymphoid aggregates than a reference number or a reference subject is treated with therapies to modulate the activities of enriched cell types: central memory T cells, Th17 T-cells, natural killer cells, B-cells, effector memory T-cells, exhausted T-cells, M2 macrophages.

In some embodiments, a subject sub-classified/stratified/identified as having the peritoneal endometriosis with a smaller number of the lymphoid aggregates (e.g., lack of relative enrichment, quantified per unit tissue size; also coined as LA^{low}) than a reference number or a reference subject is treated with an antihistamine, and not a CD20 inhibitor or an IL17 inhibitor. Therapies for this group (LA^{low}) may alternatively be or further include those that modulate the activities of enriched cell types: mast cells, MI macrophages, plasma cells, activated T-cells.

In some embodiments, the reference number used in the subclassifying patient with peritoneal endometriosis is a number (e.g., average) obtained from a plurality of subjects with peritoneal endometriosis. For example, a LA^{high} patient has signature immune cell types (central memory T cells, Th17 T-cells, natural killer cells, B-cells, effector memory T-cells, exhausted T-cells, M2 macrophages) of a larger quantity compared to an average number from a plurality of patients including both LA^{high} and LA^{low}, and a LA^{low} patient has signature immune cell types (mast cells, MI macrophages, plasma cells, activated T-cells) of a larger quantity compared to an average number from a plurality of patients including both LA^{high} and LA^{low}.

In other embodiments, a LA^{high} patient has signature immune cell types (central memory T cells, Th17 T-cells, natural killer cells, B-cells, effector memory T-cells, exhausted T-cells, M2 macrophages) of a larger quantity compared to that in a LA^{low} patient; and a LA^{low} patient has signature immune cell types (mast cells, M1 macrophages, plasma cells, activated T-cells) of a larger quantity compared to that in a LA^{high} patient.

In some embodiments, the reference number used in the subclassifying patient with peritoneal endometriosis is a predetermined threshold number. For example, in some instances, 1 or more LAs is determined as LA^{high}, and zero LAs is determined as LA^{low}, in an endometriosis tissue, e.g., a biopsy or an excised tissue that at least covers the diameter of the endometriosis. In various instances, LA^{low} refers to 0-3 (or no more than 3) LAs across at least 3 anatomic parts of a subject.

Exemplary CD20 inhibitors, including anti-CD20 antibodies, include but are not limited to ocrelizumab, rituximab, obinutuzumab, ofatumumab (KESIMPTA®), a combination of hyaluronidase (e.g., recombinant human hyaluronidase) and rituximab, divozilimab, glofitamab (an anti-CD20 & anti-CD3 bispecific antibody), ibritumomab tiuxetan, mosunetuzumab-axgb (an anti-CD20 & anti-CD3 bispecific), ripertamab, ublituximab, epcoritamab (an anti-CD20 & anti-CD3 bispecific), 304R anti-CD20 antibody (by 3SBio Inc), ABP-300 (by Abrop Corp), H-02 (by Shandong New Time Pharmaceutical Co Ltd), MIL-62 (by Beijing Mabworks Biotech Co Ltd), xacrel (by Cinnagen Co), and zuberitamab. In some embodiments, a CD20 inhibitor comprises rituximab, and it is used in one or more treatment methods disclosed herein. In some embodiments, a CD20 inhibitor comprises obinutuzumab, which is used in one or more treatment methods disclosed herein. In some embodiments, a CD20 inhibitor comprises ofatumumab, which is used in one or more treatment methods disclosed herein. In some embodiments, a CD20 inhibitor comprises ocrelizumabt, which is used in one or more treatment methods disclosed herein.

In some embodiments, CD20 inhibitors also include a CD20 targeting cell therapies such as a chimeric antigen receptor (CAR)-T cells whose antigen-specific targeting domain comprises a scFv or nanobody derived from an anti-CD20 antibody.

Exemplary IL17 inhibitor, including anti-IL17 antibodies, include but are not limited to secukinumab, ixekizumab, bimekizumab, netakimab, izokibep, SSGF-608 (by 3SBio Inc), vunakizumab, and xeligekimab. In some embodiments, an IL17 inhibitor comprises secukinumab, which is used in one or more treatment methods disclosed herein. In some embodiments, an IL17 inhibitor comprises ixekizumab, which is used in one or more treatment methods disclosed herein. In some embodiments, an IL17 inhibitor comprises bimekizumab, which is used in one or more treatment methods disclosed herein. In some embodiments, an IL17 inhibitor comprises netakimab, which is used in one or more treatment methods disclosed herein. In some embodiments, an IL17 inhibitor comprises izokibep, which is used in one or more treatment methods disclosed herein. In some embodiments, an IL17 inhibitor comprises vunakizumab, which is used in one or more treatment methods disclosed herein. In some embodiments, an IL17 inhibitor comprises xeligekimab, which is used in one or more treatment methods disclosed herein.

Exemplary anti-inflammatory agents include but are not limited to a corticosteroid, nonsteroidal anti-inflammatory drug (NSAID), anti-IL-1beta (e.g., Anakinra1), anti-TNF-a (e.g., Etanercept and Infliximab), anti-IL-6 (e.g., Tocilizumab), anti-MMP (e.g., PG-116800 and Doxycycline), macrophage modulators (e.g., phosphatidylserine-presenting liposomes), NLRP 3 inflammasome inhibitors (e.g., 16673-34-0 (5-chloro-2-methoxy-N-[2-(4-sulfamoylphenyl)ethyl]benzamide)), inflammasome antagonists (e.g., P2X7 antagonist), or anti-diabetic medications (for example, insulin (e.g., Humulin, Novolin, Humalog), metformin (e.g., Glucophage, Glucophage XR, Fortamet, Glumetza, Riomet), sulfonylureas, meglitinides, incretin mimetics, biguanides, amylinomimetic agent (e.g., Pramlintide), lipase inhibitors such as orlistat (e.g., Xenical, Alli), thiazolidinediones, Pioglitazone (e.g., Actos), Rosiglitazone (e.g., Avandia), corticosteroids such as Prednisone (e.g., Rayos), dipeptidyl peptidase-4 inhibitors, SGLT2 inhibitors, and glucagon-like peptide-1 analogs or agonists such as Exenatide (e.g., Bydureon, Byetta) and Liraglutide (e.g., Victoza)), or a combination thereof. In some embodiments, an anti-inflammatory agent comprises corticosteroid or NSAID, which is used in one or more treatment methods disclosed herein.

Exemplary antihistamines include but are not limited to cetirizine, desloratadine, fexofenadine, levocetirizine, and loratadine.

Exemplary PD1 inhibitors can be an anti-PD1 antibody or a PD1 targeting cell therapy such as a genetically engineered immune cell with a chimeric antigen receptor that has an anti-PD1 scFv or anti-PD1 nanobody. Exemplary anti-PD1 antibodies include but are not limited to Pembrolizumab, Nivolumab, Pidilizumab, Cemiplimab, Dostarlimab, Vopratelimab, Spartalizumab, Camrelizumab, Sintilimab, Tislelizumab, Toripalimab, INCMGA00012, AMP-224, AMP-514, and Acrixolimab. In some embodiments, a PD1 inhibitor comprises pembrolizumab. In some embodiments, a PD1 inhibitor comprises Nivolumab. In some embodiments, a PD1 inhibitor comprises Pidilizumab. In some embodiments, a PD1 inhibitor comprises Cemiplimab. In some embodiments, a PD1 inhibitor comprises Dostarlimab. In some embodiments, a PD1 inhibitor comprises Vopratelimab. In some embodiments, a PD1 inhibitor Spartalizumab. In some embodiments, a PD1 inhibitor comprises Camrelizumab. In some embodiments, a PD1 inhibitor comprises Sintilimab. In some embodiments, a PD1 inhibitor comprises Tislelizumab. In some embodiments, a PD1 inhibitor comprises Toripalimab.

Exemplary CTLA4 inhibitors can be an anti-CTLA4 antibody or a CTLA4 targeting cell therapy such as a genetically engineered immune cell with a chimeric antigen receptor that has an anti-CTLA4 scFv or anti-CTLA4 nanobody. Exemplary anti-CTLA4 antibodies include but are not limited to ipilimumab and tremelimumab. In some embodiments, a CTLA4 inhibitor comprises ipilimumab. In some embodiments, a CTLA4 inhibitor comprises tremelimumab.

Exemplary TIGIT inhibitors can be an anti-TIGIT antibody or a chimeric antigen-receptor-expressing immune cell derived therefrom. Exemplary TIGIT inhibitors include but are not limited to Vibostolimab, etigilimab, tiragolumab, domvanalimab, M6223, ociperlimab, and EOS884448. In some embodiments, a TIGIT inhibitor comprises vibostolimab. In some embodiments, a TIGIT inhibitor comprises etigilimab. In some embodiments, a TIGIT inhibitor comprises tiragolumab. In some embodiments, a TIGIT inhibitor comprises domvanalimab.

Exemplary LAG3 inhibitor can be an anti-LAG3 antibody or a chimeric antigen-receptor-expressing immune cell derived therefrom. Exemplary LAG3 inhibitors include but are not limited to relatlimab.

Exemplary CD86 inhibitors can be an anti-CD86 blocking monoclonal antibody, such as CD80/CD86 inhibitor, abatacept. Abatacept is a humanized fusion protein that consists of the extracellular domain of human cytotoxic T-lymphocyte-associated antigen 4 (CTLA-4) linked to a modified Fc portion of human immunoglobulin G1 (IgG1).

Exemplary NECTIN2 inhibitors can be an anti-nectin-2 antibody or antibody-drug conjugate. For example, Sim et al. in Int J Mol Sci. 2022 Oct; 23(20): 12358 describe a chimeric 1 2G1 antibody (c12G1), which is prepared by grafting the variable domain of the m1 2G1 antibody onto human IgG1, as well as an antibody-drug conjugate comprising the c1 2G1 antibody conjugated to mertansine (DM1).

Exemplary MIF inhibitors are inhibitors of macrophage migration inhibitory factor (MIF), wherein 4-iodo-6-phenylpyrimidine (4-IPP), ISO-1, and BTZO-1 are exemplary MIF inhibitor. In some embodiments, an MIF inhibitor comprises 4-IPP. In some embodiments, an MIF inhibitor comprises ISO-1.

Exemplary tumor necrosis factor (TNF) inhibitors include but are not limited to infliximab, adalimumab, etanercept, golimumab, and certolizumab.

Exemplary IL32/IL32-receptor inhibitors can be a small molecule, an antibody, a nucleotide. For example, serine protease inhibitor a-1 antitrypsin is an IL32/IL32-receptor inhibitor, which has been shown to suppress IL-32 level.

A therapy to down-regulate central memory T cells, Th17 T-cells, natural killer cells, B-cells, effector memory T-cells, exhausted T-cells, and/or M2 macrophages can be a small molecule, a polypeptide, and/or a polynucleotide, which reduces the activity and/or viability of respective cell types. For Th17 cells we can refer to the anti IL17A therapies (eg Taltz, Cosentyx)

A therapy to down-regulate mast cells, MI macrophages, plasma cells, and/or activated T-cells can be a small molecule, a polypeptide, and/or a polynucleotide, which reduces the activity and/or viability of respective cell types.

For example, a therapy to down-regulate Th17 cells include but are not limited to anti-IL17A therapies such as TALTZ (ixekizumab) and/or COSENTYX (secukinumab), and/or donepezil. Donepezil might also affect Th1 cells.

For example, a therapy to down-regulate mast cells include but are not limited to antihistamines (e.g., Allegra/Fexofenadine, Axid/Nizatidine), leukotriene inhibitors (e.g., Singulair/Montelukast), mast cell stabilizers (e.g. Cromolyn sodium, Ketotifen); or masitinib, lodoxamine, pemirolast, cromolyn sodium, olopatadine, ketotifen, epinastine, azelastine, alcaftadine.

For example, a therapy to down-regulate macrophages include but are not limited to chemokine inhibitors (e.g., Carlumab and others), CSF1R inhibitors (e.g., small molecule or antibodies), Anti-CD47/SIRPalpha antibodies, TLR agonists (e.g. targets TLR3 and TLR7), other small molecules (e.g., Guanabenz and Losartan, ibudilast and iguratimod, prednisone, trabectedin, lurbinectedin).

For example, a therapy to down-regulate exhausted T cells include but are not limited to PD1/PDL1 blocking therapies, such as PD1 inhibitors and/or PDL1 inhibitors; and/or CTLA4 blocking therapies (CTLA4 inhibitors). Exemplary PDL1 inhibitors include but are not limited to atezolizumab, avelumab, and/or durvalumab.

For example, a therapy to down-regulate plasma cells include but are not limited to proteasome inhibitors (Bortezomib, carfilzomib, ixazomib, Daratumumab) and/or therapies inhibiting IgG1 production, e.g., Elotuzumab, lenalidomide and/or dexamethasone.

An exemplary IL1A/B inhibitor is Anakinra.

For example, a therapy to down-regulate B-cells include but are not limited to antibodies targeting CD20 (e.g., rituximab), antibodies targeting CD19 (e.g., Inebilizumab), BAFF/APRIL inhibitors (e.g. Tabalumab, Belimumab, Telitacicept), BTK inhibitors (Tolebrutinib and Evobrutinib).

Methods for detecting the expression of nucleic acids (e.g., mRNA) by the genes described herein are well known in the art. Analysis of nucleic acids can be achieved using routine techniques based on hybridization to a nucleic acid sequence that is complementary to a portion of the gene's coding sequence. For example, nucleic acid binding molecules such as probes, oligonucleotides, oligonucleotide arrays, and primers can be used in assays to detect differential RNA expression in patient samples, e.g., RT-PCR. In one embodiment, RT-PCR is used according to standard methods known in the art. In another embodiment, PCR assays such as TAQMAN® assays, available from, e.g., Applied Biosystems, can be used to detect nucleic acids and variants thereof. In other embodiments, qPCR can be used to detect nucleic acids. Reagents that bind to selected biomarkers can be prepared according to methods known to those of skill in the art or purchased commercially. Applicable PCR amplification techniques are described in Ausubel et al., Short Protocols in Molecular Biology, 5th Edition, Wiley, 2002, and Innis et al., PCR Protocols, Academic Press, 1990. General nucleic acid hybridization methods are described in Anderson, “Nucleic Acid Hybridization,” BIOS Scientific Publishers, 1999.

Methods for detecting proteins expressed by the genes in the classifier are well known in the art. For example, antibody reagents can be used to detect protein expression levels of the genes of the classifiers in patient samples using any of a number of immunoassays known to those skilled in the art. Immunoassay techniques and protocols are generally described in Price and Newman, “Principles and Practice of Immunoassay,” 2nd Edition, Grove's Dictionaries, 1997; and Gosling, “Immunoassays: A Practical Approach,” Oxford University Press, 2000. A variety of immunoassay techniques, including competitive and non-competitive immunoassays, can be used. See, e.g., Self et al., Curr. Opin. Biotechnol., 7:60-65 (1996). The term immunoassay encompasses techniques including, without limitation, enzyme immunoassays (EIA) such as enzyme multiplied immunoassay technique (EMIT), enzyme-linked immunosorbent assay (ELISA), IgM antibody capture ELISA (MAC ELISA), and microparticle enzyme immunoassay (MEIA); capillary electrophoresis immunoassays (CEIA); radioimmunoassays (RIA); immunoradiometric assays (IRMA); fluorescence polarization immunoassays (FPIA); and chemiluminescence assays (CL). If desired, such immunoassays can be automated. Immunoassays can also be used in conjunction with laser induced fluorescence. See, e.g., Schmalzing et al., Electrophoresis, 18:2184-93 (1997); Bao, J. Chromatogr. B. Biomed. Sci., 699:463-80 (1997). Liposome immunoassays, such as flow-injection liposome immunoassays and liposome immunosensors, are also suitable for use in the present invention. See, e.g., Rongen et al., J. Immunol. Methods, 204:105-133 (1997). In addition, nephelometry assays, in which the formation of protein/antibody complexes results in increased light scatter that is converted to a peak rate signal as a function of the marker concentration, are suitable for use in the methods of the present invention. Nephelometry assays are commercially available from Beckman Coulter (Brea, Calif. ; Kit #449430) and can be performed using a Behring Nephelometer Analyzer (Fink et al., J. Clin. Chem. Clin. Biochem., 27:261-276 (1989)).

Autoantibodies are antibodies that react with self-antigens. Techniques to detect autoantibodies include tissue-based assays, protein-based assays and cell-based assays (CBA). With a known antigen, autoantibodies can be detected using other antigen-specific methods based on recombinant proteins and CBA using transfected cells expressing the protein in their cell membranes. Further description of cell-based assays in detecting autoantibodies is provided in Mult Scler Relat Disord. 2020 Feb: 38:101858.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

We collected 54 samples from a total of 21 patients (17 patients with endometriosis and 4 patients without endometriosis), and of the 54 samples, 32 were peritoneal endometriosis specimens from 12 patients, 8 ovarian endometriomas from 7 patients, 10 eutopic endometrium samples (7 from patients with endometriosis and 3 from patients without endometriosis), and 4 uninvolved ovary tissues (2 from patients with endometriosis and 2 from controls). All these specimens were profiled using droplet-based scRNA-seq. Single cell RNA sequencing was applied to >370,000 cells from endometriosis, eutopic endometrium, unaffected ovary and endometriosis-free peritoneum. All specimens were subjected to pathology review and genotyping for somatic variants in commonly mutated genes. Specific techniques include integrating normalized scRNA-seq data using Harmony. In total, 114 clusters were identified, and cell types were annotated using a systematic pipeline for semi-supervised cell-type assignment. (Fonseca, et al., Nature Genetics, vol. 55, February 2023, 255-267.) First, genes overexpressed in each cluster were identified (log₂ fold change (FC)=0.2; P<0.05) and a set of rules were defined for cell-type identification based on expression of canonical cell-type-specific markers, taking into consideration known hierarchies between markers, for example, ACTA2 in the absence of other fibroblast markers denotes smooth muscle cells, but when coexpressed with fibroblast markers denotes activated fibroblasts. For 108 of 114 clusters it was possible to assign cell identities using this approach.

For the six clusters that did not overexpress canonical marker genes for any cell type, pairwise correlations between all clusters were calculated and cell identities assigned based on the most correlated cluster. Correlation values for cell-type assignment ranged from 0.55 to 0.89 (Pearson correlations) and were significantly higher compared with random pairwise correlations (average random correlation r=0.007; P<0.001), providing confidence that cell-type assignment can be achieved using this approach. Gene expression within each cell type was highly correlated when fresh and cryopreserved specimens were compared.

CODEX, spatial ATAC sequencing and spatial transcriptomics was conceived for being performed on LA^{high} and LA^{low} peritoneal endometriosis to characterize the immune composition of LAs and endometriosis devoid of LAs. NicheNet was conceived for use in mapping cell-cell communications in LA^{high} and LA^{low} peritoneal endometriosis.

To test whether lymphoid aggregates have prognostic significance in endometriosis. We conceived to collect longitudinal data for patients with endometriosis in a clinical study and to use multicolor immunohistochemistry to stain LAs in the longitudinal cohort and perform image analysis to create spatial maps of LA density and distances to endometriosis foci. We also conceived to test the associations between LA presence and recurrence of endometriosis-associated symptom, to determine the prognostic significance of lymphoid aggregates in endometriosis.

Surgical and pathologic review: Patients were evaluated by the surgeon, with a detailed history and physical taken as well as all imaging reviewed, resulting in indication for surgical management. Surgery was performed either laparoscopically or robotically, with identification and excision of any obvious or suspected lesions, using either ultrasonic or monopolar energy. Wide margins were attempted for each excision. For example, a lesion in the ovarian fossa would lead to the full peritoneum in the ovarian fossa being removed. Deep infiltrating endometriosis resections were performed until normal anatomy was restored, leaving the endometriosis lesion intact. Ureterolysis and mobilization of the rectosigmoid colon were performed when necessary. Each area of excision was individually labeled and sent to the pathology laboratory for routine processing and collection of tissue for research. All patients were born with gynecologic reproductive organs. Tissues were bisected, with half processed into individual viable cells, and half formalin-fixed and paraffin-embedded (FFPE).

Tissue processing: Human endometriosis, endometrial or ovarian tissues were placed in sterile serum-free MEM at 4° C. and transferred to the tissue culture laboratory. Tissues were minced into ~1-2-mm pieces and digested with 1×Collagenase/Hyaluronidase (STEMCELL Technologies) and 100 μg ml⁻¹ DNase I (Sigma Aldrich) in 7 ml of serum-free MEM. The sample was incubated at 37° C. with constant rotation for 90 min. The supernatant was collected, and the cell suspension was spun at 300g for 10 min at 4° C. To lyse red blood cells, the cell pellet was resuspended in a red blood cell lysis buffer (0.8% NH₄Cl, 0.1% KHCO₃, pH 7.2) and incubated for 10 min at room temperature. Cell suspensions were spun again at 300 g for 10 min at 4° C. and the cell pellet was resuspended in PBS, or, if >5% dead cells were observed by trypan blue staining, cells were resuspended in dead cell removal buffer (Miltenyi Biotech) and dead cell removal was performed according to the manufacturer's instructions. Remaining cells were used directly for scRNA-seq or were frozen in 90% fetal bovine serum with 10% dimethylsulfoxide.

Identification of major cell types: To define the major cell type for each cluster we divided our procedures in two steps. In step one we performed differential expression analysis (one versus all) using MAST, implemented in the FindAllMarker and FindMarker functions in Seurat. Then, we checked the presence of the following marker genes for global annotation of cell types that were differentially expressed at log₂ FC 0.2 and adjusted P value 0.05: epithelial cells (EPCAM, KRT8, KRT18, KRT19, KRT7, KRT10), mesenchymal cells (DCN, COL11A2, FAP, PDGFRA, COL11A1, COLIAI, PDGFRB), myeloid cells (LYZ, CD14, MME, CIQA, CLEC10A), endothelial cells (CLDN5, PECAMI, CD34, ESAM), plasma cells (JCHAIN plus CD79A), B cells (JCHAIN), smooth muscle cells (ACTA2), mast cells (TPSB2), erythrocytes (HBB, GYPA), T cells (CD2, CD3D, CD3E, CD3G, CD8A, CCL5) and natural killer cells (TYROBP, FCGR3A). For each cluster we build a matrix of DEGs by normalizing each count with the total markers in each cell type. To assign the cell type based on the matrix of DEG counts we applied the following rules. First, clusters that only had cell-type-specific genes for one cell type contained within the DEG list were assigned to the corresponding cell type. If the cluster i had >35% of the cells expressing at least one keratin gene and the average of scaled expression was greater than 1, then the epithelial cell type was assigned to cluster i. If the cluster i had multiple cell-type markers contained within the DEG list, we first checked whether ACTA2 was expressed, and if so we checked whether the proportion of mesenchymal markers was greater than 25%, and then assigned the cluster i as mesenchymal cells or otherwise as smooth muscle cells. In instances where we had multiple markers but no ACTA2, we then checked which marker had the maximum proportion and assigned the correspondent cell type to cluster i, or if the multiple markers had the same proportion, we skipped the assignment for cluster i. Clusters with no counts for marker genes were also not assigned a cell type with this decision tree.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of.”

1. A method of treating a subject in need thereof, comprising:
administering one or more of a cluster of differentiation 20 (CD20) inhibitor, an interleukin-17 (IL17) inhibitor, an anti-inflammatory agent, and a therapy to down-regulate central memory T cells, T helper 17 (Th17) T-cells, natural killer cells, B-cells, effector memory T-cells, exhausted T-cells, and/or M2 macrophages to a subject indicated as having peritoneal endometriosis with a high density of lymphoid aggregates (LA^{high}), or
administering an antihistamine and/or a therapy to down-regulate mast cells, M1 macrophages, plasma cells, and/or activated T-cells to a subject indicated as having peritoneal endometriosis with a low density of lymphoid aggregates (LA^{low}),
wherein the subject is indicated as having the peritoneal endometriosis with LA^{high} according to an assessment method of claim 17, wherein the number of lymphoid aggregates per unit tissue size in a biological sample of the subject is higher compared to an average lymphoid aggregate number per unit tissue size in a plurality of reference subjects with peritoneal endometriosis, optionally the plurality of subjects being 10 or more subjects, and/or numbers of central memory T-cells, natural killer (NK) cells, Th17 T cells, effector memory CD8 T cells, Th2 T-cells, exhausted CD8 T-cells and/or macrophages M2 cells per unit tissue size in the biological sample of the subject are higher compared to an average number per unit tissue size of respective cells in the plurality of reference subjects with peritoneal endometriosis; and
wherein the subject is indicated as having the peritoneal endometriosis with LA^{low} according to the assessment method of claim 17. wherein the number of lymphoid aggregates per unit tissue size in the biological sample of the subject is lower compared to the average lymphoid aggregate number per unit tissue size in the plurality of reference subjects with peritoneal endometriosis, and/or numbers of M1 macrophages, mast cells, and/or plasma cells per unit tissue size in the biological sample of the subject are higher compared to the average number per unit tissue size of respective cells in the plurality of reference subjects with the peritoneal endometriosis.

2. A method of treating a subject in need thereof, comprising:
administering one or more of a CD20 inhibitor, an IL17 inhibitor, an anti-inflammatory agent, and optionally further administering a programmed cell death protein 1 (PD1) inhibitor, a cytotoxic T-lymphocyte associated protein 4 (CTLA4) inhibitor, a T cell immunoreceptor with immunoglobulin and ITIM domain (TIGIT) inhibitor, a lymphocyte activation gene 3 (LAG3) inhibitor, a CD86 inhibitor, a nectin-2 inhibitor, a macrophage migration inhibitory factor (MIF) inhibitor, and a tumor necrosis factor (TNF) inhibitor, to a subject indicated as having endometrioma,
wherein the subject is detected in a biological sample comprising endometrial-type tissue, according to a method of claim 15, with: the higher number of exhausted CD8 T-cells and/or higher number of plasma cells, per unit tissue size, compared to that in a specimen of peritoneal endometriosis, eutopic endometrium, or uninvolved ovary, and the lower number of lymphoid aggregates and/or lower number of B cells, per unit tissue size, compared to the specimen of peritoneal endometriosis, eutopic endometrium, or uninvolved ovary, and/or
wherein the subject is detected with presence of endometrioma in ovary optionally via laparoscopy, ultrasound, or magnetic resonance imaging (MRI).

3. (canceled)

4. The method of claim 2, wherein the exhausted CD8 T-cells in the biological sample are positive for all or one or more of PD-1, CTLA4, TIGIT, V-domain Ig suppressor of T cell activation (VISTA), and LAG3, and are negative for T cell immunoglobulin and mucin domain-containing protein 3 (TIM3); wherein the macrophages in the biological sample are M2 macrophages or are positive for all or one or more of IL10, V-set immunoglobulin-domain-containing 4 (VSIG4), regulator of G protein signaling 1 (RGS1), Lymphatic Vessel Endothelial Hyaluronan Receptor-1 (LYVE1), thrombospondin-1 (THBS1), and heparin-binding EGF-like growth factor (HBEGF); and/or wherein the B-cells in the biological sample have a lower expression level of membrane spanning 4-domains A 1 (MS4A1) and/or CD79A compared to B-cells in the specimen of peritoneal endometriosis, eutopic endometrium, or uninvolved ovary.

5. The method of claim 1, wherein the method comprises performing single cell RNA sequencing to measure expression levels in single cells, and/or wherein the method comprises performing histopathologic assays, optionally including immunohistochemistry, standard histologic stains, and/or non-standard histologic stains.

6. The method of claim 2, wherein the subject is detected with the higher number of the exhausted CD8 T-cells, and the exhausted CD8 T-cells are positive for all or one or more of PD-1, CTLA4, TIGIT, VISTA, and LAG3, and negative or undetectable for TIM3.

7. A method of treating a subject in need thereof, comprising: administering an endometriosis therapy to a subject detected according to the method of claim 16 with:
the higher number of myeloid cells and/or higher number of B cells, per unit tissue size in a biological sample of the subject, compared to respective number per unit tissue size in a specimen of eutopic endometrium, or unaffected ovary, and/or
the higher expression level in macrophages of all or one or two of insulin like growth factor 1 (IGF1), embigin (EMB), and transforming growth factor beta-1 (TGFB1) and/or a higher expression level in B cells of MS4A1, CD79A, or both, compared to the specimen of eutopic endometrium, or unaffected ovary,
wherein the endometriosis therapy comprises one or more of a CD20 inhibitor, an IL17 inhibitor, an anti-inflammatory agent, a therapy to down-regulate central memory T cells, Th17 T-cells, natural killer cells, B-cells, effector memory T-cells, exhausted T-cells, and/or M2 macrophages, an antihistamine, and/or a therapy to down-regulate, mast cells, M1 macrophages, plasma cells, and/or activated T-cells; or the endometriosis therapy comprises a procedure to remove endometriosis tissue;
wherein the myeloid cells comprise mast cells, macrophages, or both.

8. The method of claim 7, wherein the subject in need thereof has peritoneal endometriosis without endometrioma.

9. The method of claim 7, wherein the subject in need thereof has peritoneal endometriosis with endometrioma.

10. (canceled)

11. The method of claim 7, wherein the subject is detected in a blood sample with a higher level of IL17, IL32, IL1A, IL1B, SERPINEB3 autoantibody, MTUS2 autoantibody, and/or CCL7 autoantibody than a reference subject without endometriosis.

12. The method of claim 7, wherein the subject has endometriosis,
and the subject has been detected in the biological sample with the higher number of myeloid cells per unit tissue size,
and wherein the biological sample comprises a biopsy containing endometriosis tissue and optionally surrounding tissue within 1, 2, 3, 4, or 5 cm from the border of the endometriosis tissue.

13. The method of claim 12, wherein the subject has peritoneal endometriosis.

14. The method of claim 12, wherein the subject has endometrioma.

15. A method of detecting endometrioma in a subject, comprising:
detecting, in a biological sample comprising endometrial tissue of the subject:
a higher number of exhausted CD8 T-cells and/or a higher number of plasma cells, per unit tissue size, compared to respective number of a reference subject with peritoneal endometriosis in the absence of endometrioma, a reference subject with eutopic endometrium, or an uninvolved ovary tissue of the subject or of either reference subject,
a lower number of lymphoid aggregates and/or a lower number of B cells, per unit tissue size, compared to that of the reference subject with peritoneal endometriosis in the absence of endometrioma, and/or
a lower number of myeloid cells, per unit tissue size, than that of the reference subject with peritoneal endometriosis in the absence of endometrioma, optionally the number of the myeloid cells being higher compared to that of the reference subject with eutopic endometrium or compared to the uninvolved ovary tissue;
and optionally:
wherein the exhausted CD8 T-cells are positive for all or one or more of PD-1, CTLA4, TIGIT, VISTA, and LAG3, and optionally negative for TIM3,
wherein macrophages in the biological sample are in the M2 state or are positive for all or one or more of IL10, VSIG4, RGS1, LYVE1, THBS1,and HBEGF, and/or
wherein B-cells in the biological sample have a lower expression level of MS4A1, CD79A, or both, compared to B-cells of a subject with peritoneal endometriosis but not endometrioma or a subject with eutopic endometrium.

16. A method of detecting peritoneal endometriosis in a subject, comprising:
detecting, in a biological sample comprising endometrial tissue of the subject:
a higher number of myeloid cells and/or a higher number of B cells, per unit tissue size, compared to respective number per unit tissue size in a reference subject with endometrioma, a reference subject with eutopic endometrium, or an uninvolved ovary tissue of the subject or of either reference subject,
a higher number of lymphoid aggregates, per unit tissue size, compared to that of the reference subject with endometrioma, and/or
a higher expression level in macrophages of all or one or two of IGF1, EMB, and TGFB1 and/or a higher expression level in B cells of MS4A1, CD79A, or both, compared to that in the reference subject with endometrioma,
wherein the subject does not have or is not detected with endometrioma.

17. A method of assessing peritoneal endometriosis in a subject, comprising steps of:
quantifying a number of lymphoid aggregates per unit tissue size in a biological sample comprising biopsy of a tissue with suspected endometriosis or of an endometrial tissue of the subject, and
if the number of the lymphoid aggregates is larger than a reference number, further detecting a higher number of all or one or more of Th17 T-cells, NK cells, B cells, effector memory CD8 T cells, Th2 T-cells, exhausted CD8 T-cells, macrophages M2 cells, and cytotoxic CD8 T-cells, per unit tissue size, in the biological sample compared to respective cell numbers detected in a reference subject whose number of the lymphoid aggregates is smaller than the reference number, or
if the number of the lymphoid aggregates is smaller than the reference number, further detecting a higher number of all or one or more of macrophages M1, mast cells, and plasma cells per unit tissue size in the biological sample, compared to respective cell numbers detected in a reference subject whose number of the lymphoid aggregates is larger than the reference number.

18. The method of claim 17, wherein the subject does not have endometrioma.

19. A method of classifying or staging endometriosis in a subject in need thereof, comprising:
obtaining a biological sample comprising biopsy of a tissue with suspected endometriosis or of an endometrial tissue of the subject, and
measuring in the biological sample one or more of:
a number of exhausted CD8 T-cells,
a number of plasma cells and/or a number of B-cells,
a number of myeloid cells, and
a number of lymphoid aggregates,
wherein a higher number of the exhausted CD8 T-cells, a higher number of the plasma cells, a lower number of the B-cells, a lower number of the myeloid cells, and a lower number of the lymphoid aggregates, per unit tissue size, compared to respective numbers in a reference subject with peritoneal endometriosis but not endometrioma. indicates that the subject has endometrioma or that the endometriosis is at an endometrioma stage, and wherein a lower number of the exhausted CD8 T-cells, a lower number of the plasma cells, a higher number of the B cells, a higher number of the myeloid cells, and a higher number of the lymphoid aggregates, per unit tissue size, compared to respective numbers in a reference subject with endometrioma, indicates that the subject has peritoneal endometriosis but not endometrioma or that the endometriosis is at a peritoneal stage.

20. (canceled)

21. The method of claim 19, further comprising measuring the number of lymphoid aggregates in the biological sample;
wherein the subject measured as having the peritoneal endometriosis with a larger number of the lymphoid aggregates per unit tissue size than a reference number is treated with one or more of a CD20 inhibitor, an IL 17 inhibitor, an anti-inflammatory agent, and/or a therapy to down-regulate central memory T cells, Th17 T-cells, natural killer cells, B-cells, effector memory T-cells, exhausted T-cells, and/or M2 macrophages,
wherein the subject measured as having the peritoneal endometriosis with a smaller number of the lymphoid aggregates per unit tissue size than the reference number is treated with an antihistamine and/or a therapy to down-regulate mast cells, MI macrophages, plasma cells, and/or activated T-cells, and
wherein the reference number is an average number per unit tissue size in a plurality of subjects with peritoneal endometriosis, optionally the plurality of subject having peritoneal endometriosis in the absence of endometrioma and the plurality of subject being 10 or more humans.

22. (canceled)

23. (canceled)

24. (canceled)

25. A method for stratifying, or monitoring progression of peritoneal endometriosis in a subject, comprising:
performing the steps in the method of claim 17,
wherein the quantification measures a higher number of lymphoid aggregates per unit tissue size compared to an average lymphoid aggregate number per unit tissue size in a plurality of reference subjects with peritoneal endometriosis, and/or
measures a higher number of central memory T-cells, NK cells, Th17 T cells, effector memory CD8 T cells, Th2 T-cells, exhausted CD8 T-cells and/or macrophages M2 cells, per unit tissue size, compared to an average number per unit tissue size of respective cells in the plurality of reference subjects with peritoneal endometriosis,
so as to indicate that the subject has a high number of lymphoid aggregates and/or select the subject for a therapy comprising a CD20 inhibitor, an IL17 inhibitor, an anti-inflammatory, or a therapy to down-regulate central memory T cells, Th17 T-cells, natural killer cells, B-cells, effector memory T-cells, exhausted T-cells, and/or M2 macrophages; or
wherein the quantification measures a lower number of lymphoid aggregates per unit tissue size compared to the average lymphoid aggregate number per unit tissue size in the plurality of reference subjects with peritoneal endometriosis, and/or
measures in the biological sample of the subject a higher number of M1 macrophages, mast cells, and/or plasma cells per unit tissue size, compared to the average number per unit tissue size of respective cells in the plurality of reference subjects with the peritoneal endometriosis,
so as to indicate that the subject has a low number of lymphoid aggregates and/or select the subject for a therapy comprising antihistamine, or a therapy to down-regulate mast cells, MI macrophages, plasma cells, and/or activated T-cells.

26. (canceled)

27. A method for treating a subject with endometriosis, comprising:
administering a therapy to the subject detected in a biological sample with a higher number of IL32-positive T cells per unit tissue size, compared to that in an uninvolved ovary tissue of the same subject or with eutopic endometrium of a reference subject, wherein the therapy comprises one or more of (i) an IL32/IL32-receptor inhibitor, (ii) a therapy to down-regulate resident memory T cells, central memory T cells, Th17 T-cells, natural killer cells, effector memory T-cells, exhausted T-cells, and/or activated T-cells, and (iii) removal of endometriosis tissue,
wherein the biological sample comprises an endometriosis tissue and optionally further comprising surrounding tissue within 1, 2, 3, 4, or 5 cm from the border of the endometriosis tissue.

28. The method of claim 27, wherein the higher number of IL-32 positive T cells per unit tissue size is compared to an endometriosis-free biopsy of the subject.
